(19) United States
(12) Patent Application Publication
(10) Pub. No.: US 2024/0318249 A1
(43) Pub. Date: Sep. 26, 2024

(54) METHODS OF USING miR-3075-5P TO IMPROVE INSULIN SENSITIVITY AND COMPOSITIONS THEREFOR

(71) Applicant: The Regents of the University of California, Oakland, CA (US)

(72) Inventors: Wei Ying, San Diego, CA (US); Jerrold M. Olefsky, Solana Beach, CA (US)

(21) Appl. No.: 18/564,608

(22) PCT Filed: May 25, 2022
(86) PCT No.: PCT/US2022/030872

§ 371 Date: Nov. 27, 2023

Related U.S. Application Data
(60) Provisional application No. 63/195,678, filed on Jun. 1, 2021.
(60) Provisional application No. 63/202,146, filed on May 28, 2021.

(51) Int. Cl. C12Q 1/6883; C12Q 1/6869

(52) U.S. Cl. CPC C12Q 1/6883; C12Q 1/6869; C12Q 2600/136; C12Q 2600/178

(57) ABSTRACT

Described herein are compositions comprising microRNAs that can increase insulin sensitivity, reduce obesity, and reduce inflammatory responses associated with obesity.

### CROSS-REFERENCE TO RELATED APPLICATION

This application claims the priority of U.S. provisional application Ser. No. 63/202,146, filed May 28, 2021, and U.S. provisional application Ser. No. 63/195,678, filed Jun. 1, 2021, the disclosures of which are incorporated herein by reference in their entireties.

### Incorporation by Reference of Sequence Listing Provided as Text File

A Sequence Listing is provided herewith as a text file, “2241308.txt” created on May 24, 2022 and having a size of 1,715 bytes. The contents of the text file are incorporated by reference herein in their entirety.

### FEDERAL FUNDING

This invention was made with government support under DK063491, DK101395, and DK115998 awarded by National Institutes of Health. The government has certain rights in the invention.

### BACKGROUND

Obesity-induced insulin resistance is a key feature of the prediabetic state, and when subsequent β-cell dysfunction emerges, the 2 metabolic defects combine, leading to the appearance of frank Type 2 Diabetes (T2DM) (Kahn et al., 2006; Lee et al., 2018; Roden & Shulman, 2019). Insulin resistance and obesity are intricately involved in the etiology of a very common liver disease termed Non-Alcoholic Steatohepatitis (NASH) a precursor to cirrhosis with severe liver dysfunction.

While a number of antidiabetic therapeutics are available, many patients are still unable to attain adequate glucose control. Therefore, there is a large unmet medical need for new therapeutics to treat this disorder. This is particularly true with respect to insulin sensitization. Thus, although insulin resistance is a main metabolic defect in these patients, the only class of insulin sensitizers currently available is thiazolidinediones, which are infrequently used because of side effects (Nesto et al., 2004). New insulin-sensitizing therapeutics are needed.

### SUMMARY

Methods and compositions are described here that enhance insulin sensitivity and reduce obesity in a mammal that include an effective amount of a composition having at least one nucleic acid, each with a sequence that includes a seed region of miRNA-3075-5p. In some cases, the methods and compositions can also include agents that inhibit miR-434-3p, for example a nucleic acid that is complementary to miR-434-3p.

As described herein, miR-3075-5p is a RNA molecule that can significantly improve insulin sensitivity. As disclosed herein, by suppressing the expression of Fa2h, miR-3075-5p can enhance insulin sensitivity and serve as a treatment for obesity-related insulin resistance, type 2 diabetes and NASH. The importance of insulin resistance as an underlying mechanism causing NASH makes the insulin sensitizing effects of miR-3075-5p a significant benefit in the prevention and treatment of NASH, as well as other diseases such as insulin resistance and obesity. A composition comprising miR-3075-5p is useful to prevent, inhibit or treat diseases including but not limited to obesity-related insulin resistance, type 2 diabetes and NASH.

### BRIEF DESCRIPTION OF THE FIGURES

FIG. 1A-1L illustrate that hepatocyte-derived exosomes (Exos) from early stage obese mice enhance insulin sensitivity. FIG. 1A graphically illustrates that extracellular vesicles (EVs), which include exosomes (Exos), are produced by lean normocaloric (left bar), 4 week high fat diet (HFD, middle bar), and 16 week HFD (right bar) fed wild type mouse hepatocytes. As shown, more Exos were produced by 4 week HFD and 16 week HFD mouse hepatocytes than from hepatocytes of mice maintained on normocaloric chow-fed lean mice. FIG. 1B graphically illustrates glucose tolerance tests of obese mice (12 week HFD/obese WT mice) after 6 weeks treatment with 4 week HFD hepatocyte Exos (lowest line). Lean Exos (middle line) were also administered to a subset of obese mice. HFD Con mice (top line) were obese but did not receive Exos. As illustrated, the 4 week HFD hepatocyte Exos reduced glucose levels in the obese mice relative to the HFD Con and Lean Exos groups of mice. FIG. 1C graphically illustrates insulin tolerance tests of obese mice (12 week HFD/obese WT mice) after 6 weeks treatment with 4 week HFD hepatocyte Exos (lowest line). Lean Exos (top line) were also administered to a subset of obese mice. HFD Con mice (middle line) were obese but did not receive Exos. As illustrated, the 4 week HFD hepatocyte Exos increased insulin sensitivity (reduced glucose levels) in the obese mice relative to the HFD Con and Lean Exos groups of mice. FIG. 1D graphically illustrates the glucose infusion rate (GIR) during hyperinsulinemic-euglycemic clamp studies in control mice (HFD Con, left bar) and in 12 week HFD/obese mice intravenously injected with Exos from 4 week HFD mice (right bar). FIG. 1E graphically illustrates the insulin-stimulated glucose disposal rate (IS-GDR) in control mice (HFD con, left bar) and in 12 week HFD/obese mice intravenously injected with Exos from 4 week HFD mice (right bar). FIG. 1F graphically illustrates the hepatic glucose production (HGP) in control mice (HFD Con, left bars) and in 12 week HFD/obese mice intravenously injected with Exos from 4 week HFD mice (right bars). FIG. 1G graphically illustrates the percentage suppression of free fatty-acid levels (FFA suppression) during hyperinsulinemic-euglycemic clamp studies in control mice (HFD Con, left bar) and in 12 week HFD/obese mice intravenously injected with Exos from 4 week HFD mice (right bar). FIG. 1H illustrates insulin-stimulated protein kinase B (PKB, also called AKT) phosphorylation in liver, skeletal muscle (SKL), and epididymal fat of 18 week HFD/obese mice treated with 4 wk HFD Exos. Data are presented as mean±SEM. n=8 per group (FIG. 1B-1C). *P<0.05, **P<0.01, 532 Student's t-test. FIG. 1I graphically illustrates the effects of 4 week HFD Exos (right bars) compared to untreated control (HFD con, left bars) on proinflammatory cytokine abundance in epididymal fat of 18 week HFD mice as detected by qPCR analysis. FIG. 1J graphically illustrates the effects of 4 week HFD Exos on glucose uptake by 3T3-L1 adipocytes in chronically obese recipient HFD mice that were treated with 4 week HFD Exos. FIG. 1K graphically illustrates the effects of 4 week HFD Exos on glucose uptake by L6 myocytes in chronically obese recipient HFD mice that were treated with 4 week HFD Exos. FIG. 1L graphically illustrates the effects of 4 week HFD Exos (right 3 bars) on primary hepatocytes isolated from 12 week HFD/obese mice treated with the hepatocyte Exos from 4 week HFD mice. As illustrated 4 week HFD Exos led to enhanced insulin-mediated suppression of glucagon-stimulated glucose (GCG-Ins) production, compared to glucagon-stimulated glucose (GCG) levels from insulin-resistant obese hepatocytes treated with empty liposomes (HFD Hep, left 3 bars).

FIG. 2A-2I illustrate that miRNAs are the key cargo causing the hepatocyte Exos effects. FIG. 2A illustrates abundance of Exos production (left) and TSG101 expression (an Exos marker, right) of Exos derived from hepatocytes with and without YBX1-knockout (YBX1-KO). As illustrated, deletion of YBX1 did not significantly affect hepatocyte Exos production or the expression of Exos-associated protein markers such as TSG101. FIG. 2B graphically illustrates the effect of YBX1-KO on miR-122 in hepatocyte Exos. MiR-122 is typically highly expressed miRNA in hepatocytes, but not Exos from YBX1-KO hepatocytes. FIG. 2C graphically illustrates the effect of hepatocyte-specific YBX1-KO (upper line) on glucose tolerance after 4 weeks HFD feeding compared to wild type control (lower line). FIG. 2D graphically illustrates the effect of hepatocyte-specific YBX1-KO (upper line) compared to wild type HFD control (lower line) on insulin sensitivity after 4 weeks HFD feeding. FIG. 2E illustrates the effects of administration of HFD wild type hepatocyte Exos (left bar) compared to YBX1-KO 4 week HFD Exos (middle line) and wild type 4 week HFD Exos (right bar) on the body weights of 18 week HFD/obese recipient mice. FIG. 2F illustrates glucose tolerance tests in 18 week-HFD/obese mice treated with either 4 week HFD Exos (lower line) or 4 week HFD YBX1-KO Exos (middle line), compared to HFD control (upper line). FIG. 2G illustrates insulin tolerance tests in 18 week HFD/obese mice treated with either 4 week HFD Exos (lower line) or 4 week HFD YBX1-KO Exos (middle line at 60-90 min) compared to HFD Control (top line at 60-90 min). FIG. 2H illustrates the effects of 4 week HFD YBX1-KO Exos (right bars) on glucose uptake in 3T3-L1 adipocytes compared to control (left bars). FIG. 2I illustrates the effects of 4 week HFD YBX1KO Exos (right bars) on glucose uptake in L6 myocytes compared to L6 myocytes control (left bars). Data are presented as mean±SEM. n=8 per group (c-fl. *P<0.05, Student's t-test.

FIG. 3A-3G illustrate the effects of hepatocyte-specific knockout (KO) of Rab27 (a small GTPase) on Exos secretion and miRNA content of Exos. FIG. 3A illustrates the effects of Rab27-KO on hepatic Exos production. FIG. 3B illustrates miR-122 abundance within Exos derived from hepatocytes after knockout of Rab27. FIG. 3C illustrates the effects of 4 wk HFD Rab27-KO hepatocyte Exos (3 right bars) compared to control (left 3 bars) on glucose production in 16 wk HFD/obese primary hepatocytes. FIG. 3D illustrates glucose tolerance tests of hepatocyte-specific Rab27-KO mice (upper line) after 4 weeks HFD feeding compared to control (lower line). FIG. 3E illustrates insulin tolerance tests in hepatocyte-specific Rab27-KO mice (upper line) after 4 weeks HFD feeding compared to control (lower line). As illustrated, Rab27 depletion resulted in glucose intolerance and insulin resistance. FIG. 3F illustrates insulin-related phosphorylation of AKT in skeletal muscle (SKL), liver, and visceral adipose tissue (VAT) from 4 wk HFD WT and hepatocyte Rab27-KO mice. Data are presented as mean±SEM. n=8 per group (c and d). *P<0.05, Student's t-test. FIG. 3G illustrates hepatic glucose production in 4 week HFD wild type mice (left 3 bars) and 4 week HFD Rab27 knockout mice (right 3 bars). Data are presented as mean±SEM. *P<0.05, Student's t-test.

FIG. 4A-4N illustrate miR-3075 abundance and the effects of miR-3075 on insulin sensitivity. FIG. 4A illustrates miR-3075 abundance in the livers of 18 week HFD wild type recipient mice after treatment with 4 week HFD Exos (middle bar) compared to treatment with 4 week HFD Exos from YBX1-knockout mice (right) and untreated control HFD wild type (left bar). FIG. 4B illustrates miR-3075 abundance in the skeletal muscle (SKL) of 18 week HFD wild type recipient mice after treatment with 4 week HFD Exos (middle bar) compared to treatment with 4 week HFD Exos from YBX1-knockout mice (right) and untreated control HFD wild type (left bar). FIG. 4C illustrates miR-3075 abundance in the epididymal white adipose (eWAT) of 18 week HFD wild type recipient mice after treatment with 4 week HFD Exos (middle bar) compared to treatment with 4 week HFD Exos from YBX1-knockout mice (right) and untreated control HFD wild type (left bar). FIG. 4D illustrates miR-3075 abundance in lean wild type hepatocytes (left bar), 4 week HFD wild type hepatocytes (middle bar), or 16 week HFD (right bar) wild type hepatocytes as quantified by qPCR analysis. FIG. 4E illustrates hepatic glucose production in lean wild type hepatocytes (left 3 bars), 4 week HFD wild type hepatocytes (middle 3 bars), or 16 week HFD wild type hepatocytes (right 3 bars). FIG. 4F illustrates insulin-stimulated AKT phosphorylation in lean wild type hepatocytes, 4 week HFD wild type hepatocytes, or 16 week HFD wild type hepatocytes. FIG. 4G illustrates the effects of a miR-3075 antagomir (a chemically engineered oligonucleotide that blocks miR-3075 binding) on AKT phosphorylation levels in 4 week HFD hepatocytes. FIG. 4H illustrates miR-3075 abundance in key metabolic tissues of lean WT mice, 3T3-L1 adipocytes, and L6 myocytes. FIG. 4I illustrates glucose tolerance of wild type mice on a normocaloric diet (NCD, lowest line), on a 4 week HFD (middle line), and on a 16 week HFD (top line). FIG. 4J illustrates miR-3075 abundance within Exos derived from lean hepatocytes transfected without and with the miR-3075 mimic (miR-3075oe), as detected by qPCR analysis. FIG. 4K illustrates the effects of miR-3075-enriched hepatocyte (miR-3075oe) Exos on insulin-resistant 3T3-L1 adipocyte glucose uptake. FIG. 4L illustrates the levels of phosphorylated AKT in metabolic tissues of 4 weeks HFD WT mice after treatment with the miR-3075 antagomir. FIG. 4M illustrates the effects of a miR-3075 antagomir (lower line at 30 min) on glucose tolerance of lean WT mice. FIG. 4N illustrates the effects of miR-3075 antagomir (upper line at 30-90 min) on insulin sensitivity of lean WT mice. n=8 per group (FIG. 4I), n=5-6 (FIG. 4M-4N). Data are presented as mean±SEM.*P<0.05, ***P<0.001, Student's t-test.

FIG. 5A-5N illustrate that miR-3075 is an insulin sensitizer within 4 week HFD Exos. FIG. 5A illustrates hepatic glucose output of primary hepatocytes from wild type mice, where the hepatocytes were treated with a miR-3075 mimic (right 3 bars) and the results were compared to untreated control HFD primary hepatocytes (left 3 bars). FIG. 5B illustrates glucose uptake of 3T3-L1 adipocytes treated with a miR-3075 mimic (right bars), compared to untreated control (left bars). FIG. 5C illustrates glucose uptake by L6 myocytes treated with a miR-3075 mimic (right bars), compared to untreated control (left bars). FIG. 5D illustrates insulin-stimulated phosphorylated AKT levels in recipient cells after 24 hours transfection with the miR-3075 mimic. FIG. 5E illustrates glucose uptake by 3T3-L1 adipocytes after treatment with the miR-3075 mimic and/or miR-3075 antagomir. Compared to control (left 2 bars), the results show the effects of treatment with the miR-3075 mimic (third & fourth bars from the left), miR-3075 antagomir (fifth & sixth bars from the left), or a combination the miR-3075 mimic and miR-3075 antagomir (rightmost 2 bars). FIG. 5F illustrates glucose uptake by L6 myocytes after treatment with the miR-3075 mimic and/or miR-3075 antagomir. Compared to control (left 2 bars), the results show the effects of treatment with the miR-3075 mimic (third & fourth bars from the left), miR-3075 antagomir (fifth & sixth bars from the left), or a combination the miR-3075 mimic and miR-3075 antagomir (rightmost 2 bars). FIG. 5G illustrates the effects of miR-3075 antagomir treatment (3 right bars) on glucose production of hepatocytes isolated from 4 week HFD WT mice compared to untreated 4 week HFD WT hepatocytes (left 3 bars). FIG. 5H illustrates the effects of the miR-3075 antagomir on 4 week HFD Exos-induced 3T3-L1 adipocyte glucose uptake. Compared to Basal levels (4 lowest bars), insulin stimulated glucose uptake (4 tallest bars). FIG. 5I illustrates the effects of the miR-3075 antagomir on 4 week HFD Exos-induced L6 myocyte glucose uptake. Compared to Basal levels (4 lowest bars), insulin stimulated glucose uptake (4 tallest bars). FIG. 5J illustrates the effects of the miR-3075 antagomir on 4 week HFD Exos-induced obese primary hepatocyte glucose production. For each set of three bars, basal levels are shown by the left bar, GCG levels are shown by the middle bar, and GCG/insulin levels are shown by the right bar. FIG. 5K illustrates the effects of miR-3075 antagomir (top line) compared to untreated control (lower line) on glucose tolerance in 4 week HFD-fed WT mice. FIG. 5L illustrates the effects of miR-3075 antagomir (upper line) compared to control (lower line) on insulin sensitivity in 4 week HFD-fed WT mice. As shown, miR-3075 antagomir treatment of 4 week HFD-fed WT mice led to impaired glucose tolerance and insulin resistance compared to control mice treated with empty liposomes. FIG. 5M illustrates miR-3075 levels in eWAT and SKL tissues of lean (NCD, left data points) WT, 4 week HFD WT (middle data points), or hepatocyte-specific Rab27-KO mice (right data points) as detected by qPCR analysis. Data are presented as mean±SEM. *P<0.05, ***P<0.001, Student's t-test. FIG. 5N illustrates that addition of a miR-3075 mimic (right bars in left graph) modulates expression of mRNAs that harbor a miR-3075 target binding site as shown by qPCR analysis, and that miR-3075 represses FA2H expression in human HepG2 cells as detected by immunoblot (right).

FIG. 6A-6H show that Fa2h is a miR-3075 target gene that regulates cellular insulin responses. FIG. 6A illustrates FA2H abundance in metabolic tissues of 18 week HFD/obese recipient mice treated with 4 week HFD Exos. FIG. 6B illustrates luciferase activity in HEK293 cells transfected with an Fa2h 3′UTR reporter vector with miR-3075 mimic (right bar) or without miR-3075 mimic (left bar) treatment. FIG. 6C illustrates the effect of Fa2h knockdown (siRNA FA2H, right bars) on glucose uptake in 3T3-L1 adipocytes and AKT phosphorylation. To knockdown FA2H expression an antisense siRNA targeted to Fa2h mRNA was used. FIG. 6D illustrates the effect of Fa2h knockdown (siRNA FA2H, right bars) on glucose uptake in L6 myocytes and AKT phosphorylation. FIG. 6E illustrates the effect of Fa2h knockdown (siRNA FA2H, right bars) on hepatocyte glucose output and AKT phosphorylation. Data are presented as mean±SEM. *P<0.05, **P<0.01, ***P<0.001, Student's t-test. FIG. 6F illustrates miR-3075 expression in human HepG2 cells compared to lean mouse hepatocytes. FIG. 6G illustrates the effect of miR-3075 overexpression on FA2H abundance in human HepG2 cells. FIG. 6H is an image of an immunoblot, illustrating validation of FA2H knockdown in 3T3-L1 adipocytes, L6 myocytes, and obese hepatocytes.

FIG. 7A-7K illustrate that chronic obesity induces hepatocytes to secrete pathogenic exosomal miRNAs that cause tissue inflammation and insulin resistance. FIG. 7A illustrates glucose tolerance in hepatocyte-specific Rab27 knockout mice (middle line at 40 min) and in YBX1 knockout mice (lowest line at 40 min) after 16 weeks HFD feeding compared to control (top line). FIG. 7B illustrates insulin sensitivity in hepatocyte-specific Rab27 knockout mice (middle line at 30 min) and in YBX1 knockout mice (lowest line at 30 min) after 16 weeks HFD feeding compared to control (top line). FIG. 7C illustrates glucose tolerance of NCD wild type mice after 5 weeks treatment with either 16 week HFD Exos (top line) or 16 week HFD YBX1-KO Exos (middle line). FIG. 7D illustrates insulin sensitivity of NCD wild type mice after 5 weeks treatment with either 16 week HFD Exos (top line) or 16 week HFD YBX1-KO Exos (middle line at 30 min). FIG. 7E illustrates the effect of 16 week HFD Exos on insulin-stimulated phosphorylation of AKT in normocaloric diet (NCD) recipient mice. FIG. 7F illustrates the effect of activation of the NFκB pathway (phosphorylation of the P65 subunit of NFκB) in NCD recipient mice. FIG. 7G illustrates abundance of proinflammatory genes in eWAT of NCD (left bars) recipient mice after 5 weeks treatment with 16 week HFD Exos (right bars). FIG. 7H illustrates abundance of proinflammatory genes in liver of NCD (left bars) recipient mice after 5 weeks treatment with 16 week HFD Exos (right bars). Data are presented as mean±SEM. n=6 per group (FIG. 7A-7D). *P<0.05, ***P<0.001, Student's t-test. FIG. 7I illustrates glucose uptake by 3T3-L1 adipocytes (left bars) treated with 16 wk HFD Exos (right bars), illustrating that the 16 wk HFD Exos did not significantly affect glucose uptake by the 3T3-L1 adipocytes. FIG. 7J glucose uptake by L6 myocytes (left bars) treated with 16 wk HFD Exos (right bars), illustrating that the 16 wk HFD Exos did not significantly affect glucose uptake by the L6 myocytes. FIG. 7K illustrates glucose production by untreated hepatocytes (left 3 bars) and hepatocytes treated with 16 wk HFD Exos (right 3 bars), illustrating that the 16 wk HFD Exos did not significantly affect glucose uptake by hepatocytes. Data are presented as mean±SEM.

FIG. 8A-8G shows that hepatocyte-derived Exos from chronic obese mice stimulate the pro-inflammatory M1-like state in macrophages. FIG. 8A illustrates the effects of 16 week HFD Exos (middle bars) or 16 week HFD YBX1-KO Exos (right bars) on the population and activation state of CD45+ leukocytes and adipose tissue macrophages (ATMs) in normocaloric diet (NCD) WT mice. FIG. 8B illustrates activation of bone marrow derived macrocytes (BMDMs) in response to LPS (M1) after transfection with the miR-434-3p mimic (right bars). FIG. 8C illustrates activation of BMDMs in response to IL4/IL13 (M2) stimulation after transfection with the miR-434-3p mimic (right bars). FIG. 8D illustrates the effect of a miR-434-3p mimic (right bars) on proinflammatory activation of ATMs from lean mice. FIG. 8E illustrates the abundance of key genes associated with activation of HFD/obese ATMs after treatment with the miR-434-3p antagomir (right bars). Data are presented as mean±SEM. *P<0.05, **P<0.01, ***P<592 0.001, Student's t-test. FIG. 8F illustrates miR-434-3p abundance in epididymal fat of normocaloric diet (NCD) recipient mice after treatment with 16 wk HFD Exos (right bar) for 5 weeks. FIG. 8G illustrates miR-434-3p abundance in BMDMs after transfection with the miR-434-3p mimic (right bar) as measured by qPCR analysis.

### DETAILED DESCRIPTION

Compositions and methods are described herein that can enhance insulin sensitivity in a mammal. The compositions include microRNAs (miRNAs) such as miRNA-3075, miRNA-3075-5p, or combinations thereof. Also, as illustrated herein, inhibitors of miR-434 and/or miR-434-3p can reduce inflammatory responses associated with obesity, especially chronic obesity. Hence, the compositions and/or methods described herein can include one or more types of miRNA-3075, and the compositions and/or methods described herein can also include one or more types of miRNA-3075 combined with at least one inhibitor of miR-434 and/or miR-434-3p.

The inventors have demonstrated that miRNA-3075, miRNA-3075-5p, or combinations thereof are secreted in exosomes by hepatocytes and/or macrophages from obese mice. Adipose tissue macrophages (ATM) exosomes (Exos) derived from obese mice directly cause insulin resistance in vitro, and when given to lean mice, produce a state of insulin resistance and glucose intolerance. In contrast, as described hereon, ATM Exos derived from lean mice lead to improved insulin sensitivity both in vitro and in vivo. This latter finding indicates that the M2-like ATMs which are present in lean adipose tissue can secrete Exos which carry “therapeutic” mRNAs as a cargo constituent. Indeed, we have recently demonstrated that miR-690 is abundantly expressed in M2 macrophage-derived exosomes and can directly cause enhanced sensitivity in vitro in cells and reverses insulin resistance/glucose intolerance when given to obese mice in vivo (Ying et al., 2021).

The liver plays an essential role in the metabolism of glucose and lipids and must quickly adapt to differing conditions of nutrient availability by regulating the fluxes of carbohydrates and lipids (Scheja & Heeren, 2016; Turner et al., 2013). Previous studies have reported that various hepatokines, such as andropin, angiopoietin-like protein 6, and fibroblast growth factor 21 can act in an endocrine manner to enhance insulin signaling in adipose tissue and skeletal muscle in the context of obesity (Kumar et al., 2008; Gao et al., 2015; Oike et al., 2005; Ebert et al., 2009; Kharitonenkov et al., 2005; Chavez et al., 2009). In contrast, after the establishment of obesity, a set of pathogenic hepatokines, including fetuin A, hepassocin, DPP4, and selenoprotein p have been reported to exacerbate insulin resistance or chronic tissue inflammation (Ghorpade et al., 2018; Varin et al., 2019; Misu et al., 2010; Choi et al., 2013; Yang et al., 2011; Wu et al., 2016; Auberger et al., 1989; Mukhopadhyay & Bhattacharya, 2016). While some studies have reported that hepatocyte-derived extracellular vesicles (EVs) can act as paracrine molecules leading to activation of hepatic stellate cells (Povero et al., 2015; Lee et al., 2017; Chen et al., 2015; Pescador et al., 2013; Chen et al., 2019), little is known about the systemic effects of hepatocyte exosomal miRNAs on the metabolic dysfunction in obesity.

As disclosed herein, hepatocytes secrete distinct exosomal microRNAs (miRNAs) in early onset vs. chronic established obesity that modulate peripheral insulin sensitivity and glucose tolerance. Hepatocyte-derived Exos collected from 4 week HFD mice (early stage obesity) can directly enhance both in vitro and in vivo insulin sensitivity. miR-3075 is highly induced in these early onset 4 week HFD hepatocyte-derived Exos and directly causes these beneficial metabolic effects. Based on exosomal miRNA sequencing and a bioinformatics approach, we identified FA2H mRNA as a target of miR-3075. Indeed, FA2H expression is suppressed after treatment with 4 week HFD hepatocyte Exos or with a miR-3075 mimetic. In contrast to the findings with early stage 4 week HFD hepatocyte Exos, chronic established obesity in 16 weeks HFD mice leads to the secretion of hepatocyte Exos which no longer express high levels of miR-3075. Indeed, these 16 week HFD hepatocyte Exos indirectly exaggerate insulin resistance by stimulating proinflammatory macrophage responses in adipose tissue. Thus, in early stage obesity hepatocytes secrete exosomes which appear to be a compensatory response mitigating the full development of insulin resistance, whereas, in the chronic obese state, hepatocyte-derived Exos promote insulin resistance.

Exosomes (Exos) and their molecular cargos play roles in intercellular and interorgan crosstalk (Mathieu et al., 2019). In a sense, the systemic inter-organ crosstalk attributed to secreted Exos resembles an endocrine-like system. Emerging evidence has revealed that Exos-mediated intercellular communication exerts major effects on insulin resistance and glucose intolerance in the obese setting (Castano et al., 2018; Deng et al., 2009; Ferrante et al., 2015; Flaherty et al., 2019; Ying et al., 2017; Ying et al., 2021). These effects have largely been attributed to the microRNAs (miRNAs) carried within exosomes (Mori et al., 2019; Ying et al., 2017; Ying et al., 2021; Thomou et al., 2017). miRNAs negatively regulate gene expression by binding to the 3′ untranslated region (3′ UTR) of mRNA targets, leading to translational arrest or degradation (Baek et al., 2008; Bartel, 2009; Selbach et al., 2008). Exosomal miRNAs have been implicated in obesity-induced insulin resistance and glucose intolerance in both animals and humans (Mori et al., 2019). As one example, the inventors studies have demonstrated that adipose tissue macrophages (ATMs) secrete miRNA containing Exos which can enter the circulation to be taken up by distal insulin target cells, such as liver, muscle, and adipocytes (Ying et al., 2017). However, whether particular microRNAs are involved, and what those microRNAs may be, was unknown until the work performed by the inventors and described herein.

MicroRNAs are generated from primary transcripts mainly through the sequential cleavages by two enzymes, Drosha and Dicer. The mature microRNA is the result of such cleavage. The sequence of the mature microRNA, especially the ‘seed sequence’, largely determines its binding ability and specificity for target mRNAs.

A human mature miRNA-3075 has the following sequence (SEQ ID NO: 1): UGUCUGGGAGCAGCCAAGGAC. The mature mouse miRNA-3075 sequence is the same. A pre-mature, stem-loop form of miRNA-3075 has the sequence:

(SEQ ID NO: 2)
UGUGGUUUGUACUGUCUGGGAGCAGCCAAGGACAAGUUACCUCUU
GUCUUCUAUCCUUGGCCUUCCUAGGUGUCCAAGCUCACAG.

A sequence for miRNA-3075-5p is shown below as SEQ ID NO:3: UGUGGUUUGUACUGUCUGGGAGCAGCCAAGGACAAGUUA. The seed region of miRNA-3075-5p can include 5′CUGGGAG3′ or 5′GCCAAGGA3′ or the corresponding DNA sequences (5′CTGGGAG3′ or 5′GCCAAGGA3′).

Some variants or isoforms of sequences can occur amongst populations of patients or subjects. Hence, the compositions and methods described herein can include some sequence variations. For example, the miRNA-3075 can have at least 70%, 80%, e.g., at least 85%, 87%, 90%, 92%, 93%, 94%, 95%, 96%, 97%, 98% or 99% nucleic acid sequence identity to SEQ ID NO: 1, SEQ ID NO:2, or SEQ ID NO:3, as well as the corresponding DNA sequences. In one embodiment, the ribonucleotides or deoxyribonucleotides include one or more modified ribonucleotides or deoxyribonucleotides, e.g., modified phosphate linkages, modified sugars, modified nucleobases, or combinations thereof.

Also as illustrated herein inhibitors of miR-434 and/or miR-434-3p can reduce inflammatory responses associated with obesity. The following is a mature sequence for human miR-434-3p: UUUGAACCAUCACUCGACUCCU (SEQ ID NO:4). A stem-loop, pre-mature sequence for human miR-434 is shown below as SEQ ID NO:5: UCGACUCUGGGUUUGAACCAAAGCUCGACUCAUGGUUUGAACCAU UACUUAAUUCGUGGUUUGAACCAUCACUCGACUCCUGGUUCGAACC AUC. Human and mouse mature miR-434 and miR-434-3p are generally the same. Inhibitors of miR-434 and/or miR-434-3p can have a natural, synthetic, or chemically modified sequence that is complementary to miR-434 and miR-434-3p. Hence, inhibitors of miR-434 and/or miR-434-3p can be natural or modified nucleic acids such as any of the inhibitory nucleic acids described herein.

Some variants or isoforms of sequences can occur amongst populations of patients or subjects. Hence, the compositions and methods described herein can include some sequence variations. For example, the miRNA-3075 and/or miR-3075-5p can have at least 70%, 80%, e.g., at least 85%, 87%, 90%, 92%, 93%, 94%, 95%, 96%, 97%, 98% or 99% nucleic acid sequence identity or complementarity to SEQ ID NO: 1, SEQ ID NO:2, SEQ ID NO:3, as well as the corresponding DNA sequences. For example, the miR-434 and/or miR-434-3p can have at least 70%, 80%, e.g., at least 85%, 87%, 90%, 92%, 93%, 94%, 95%, 96%, 97%, 98% or 99% nucleic acid sequence identity or complementarity to SEQ ID NO:4, SEQ ID NO:5, as well as the corresponding DNA sequences. In one embodiment, the ribonucleotides or deoxyribonucleotides include one or more modified ribonucleotides or deoxyribonucleotides, e.g., modified phosphate linkages, modified sugars, modified nucleobases, or combinations thereof.

### Modified Nucleic Acids

The miRNA-3075, miRNA-3075-5p, miRNA-434 inhibitory, and/or miRNA-434-3p inhibitory nucleic acids can be made from natural nucleotides, unnatural nucleotides, natural bonds, unnatural bonds, or a combination thereof when used in the methods and compositions described herein.

The miRNA-3075, miRNA-3075-5p, miRNA-434 inhibitory, and/or miRNA-434-3p inhibitory nucleic acid may be prepared using available methods, for example, by expression from an expression vector encoding a complementary or non-complementary sequence of the miRNA-3075, miRNA-3075-5p, miRNA-434, or mir-434-3p. Alternatively, it may be prepared by chemical synthesis using naturally occurring nucleotides, modified nucleotides, or any mixture of combination thereof. In some embodiments, the miRNA-3075, miRNA-3075-5p, miRNA-434 inhibitory, and/or the mir-434-3p inhibitory nucleic acids are made from modified nucleotides or non-phosphodiester bonds. Such modified nucleotides or non-phosphodiester bonds, for example, can increase biological stability of the nucleic acids and/or to increase intracellular stability of the duplex formed between the nucleic acids and other (e.g., endogenous) nucleic acids.

For example, the miRNA-3075, miRNA-3075-5p, miRNA-434 inhibitory, and/or the mir-434-3p inhibitory nucleic acids can be peptide nucleic acids that have peptide bonds rather than phosphodiester bonds.

Naturally occurring nucleotides that can be employed in the miRNA-3075, miRNA-3075-5p, miRNA-434 inhibitory, and/or the mir-434-3p inhibitory nucleic acids include the ribose or deoxyribose nucleotides adenosine, guanine, cytosine, thymine and uracil. Examples of modified nucleotides that can be employed in the miRNA-3075, miRNA-3075-5p, miRNA-434 inhibitory, and/or the mir-434-3p inhibitory nucleic acids include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladenine, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine.

Thus, the miRNA-3075, miRNA-3075-5p, miRNA-434 inhibitory, and/or the mir-434-3p inhibitory nucleic acids described herein may include modified nucleotides, as well as natural nucleotides such as combinations of ribose and deoxyribose nucleotides. The miRNA-3075, miRNA-3075-5p, miRNA-434 inhibitory, and/or the mir-434-3p inhibitory nucleic acids and may be of same length as wild type miRNA-3075, miRNA-3075-5p, miRNA-434 or mir-434-3p described herein. The miRNA-3075, miRNA-3075-5p, miRNA-434 inhibitory, and/or the mir-434-3p inhibitory nucleic acids described herein can also be longer and include other useful sequences. In some embodiments, the miRNA-3075, miRNA-3075-5p, miRNA-434 inhibitory, and/or the mir-434-3p inhibitory nucleic acids described herein are somewhat shorter. For example, such nucleic acids can be missing up to 2 nucleotides, or missing up to 3 nucleotides, or missing up to 5 nucleotides, or missing up to 7 nucleotides, or missing up to 10 nucleotides from the 5′ or 3′ end of the natural miRNA-3075, miRNA-3075-5p, miRNA-434, or the mir-434-3p nucleic acids.

The miRNA-3075, miRNA-3075-5p, miRNA-434 inhibitory, and/or the mir-434-3p inhibitory nucleic acids can be introduced via one or more vehicles such as via expression vectors (e.g., viral vectors), via virus like particles, via ribonucleoproteins (RNPs), via nanoparticles, via liposomes, or a combination thereof. The vehicles can include components or agents that can target particular cell types, facilitate cell penetration, reduce degradation, or a combination thereof.

### Exemplary Delivery Vehicles

As described herein, miRNA-3075 and/or miRNA-3075-5p can enhance insulin sensitivity in a mammal. In addition, inhibitors of miR-434-3p can reduce inflammatory responses associated with obesity. Hence, compositions are described herein that include one or more types of nucleic acids, at least one of which includes at least a seed region of miRNA-3075. In some cases, the methods and compositions can also include agents that inhibit miR-434-3p, such as antisense or antagomirs of miR-434-3p.

Delivery vehicles for miRNA-3075/miRNA-3075-5p and/or for nucleic acid inhibitors of miR-434/miR-434-3p that can be formulated into compositions include, for example, naturally occurring polymers, microparticles, nanoparticles, and other macromolecular complexes capable of mediating delivery of a nucleic acid to a host cell. Vehicles can also comprise other components or functionalities that further modulate, or that otherwise provide beneficial properties.

A mature sequence for miRNA-3075 is UGUCUGGGAGCAGCCAAGGAC (SEQ ID NO:1). A stem-loop form of miRNA-3075 has the sequence: UGUGGUUUGUACUGUCUGGGAGCAGCCAAGGACAAGUUACCUCUU GUCUUCUAUCCUUGGCCUUCCUAGGUGUCCAAGCUCACAG (SEQ ID NO:2). A sequence for miRNA-3075-5p is shown below as SEQ ID NO:3: UGUGGUUUGUACUGUCUGGGAGCAGCCAAGGACAAGUUA. Any of the SEQ ID NO: 1-3 of the miRNA-3075 nucleic acids, or combinations thereof, can be included in the compositions and used in the methods described herein.

Also as illustrated herein inhibitors of miR-434 and/or miR-434-3p can reduce inflammatory responses associated with obesity. The following is a mature sequence for human miR-434-3p: UUUGAACCAUCACUCGACUCCU (SEQ ID NO:4). A stem-loop, pre-mature sequence for human miR-434 is shown below as SEQ ID NO:5: UCGACUCUGGGUUUGAACCAAAGCUCGACUCAU GGUUUGAACCAUUACUUAAUUCGUGGUUUGAACCAUCACUCGACU CCUGGUUCGAACCAUC. Human and mouse mature miR-434 and miR-434-3p are generally the same. Inhibitors of any of the SEQ ID NO:4-5 miRNA-434 nucleic acids, or combinations thereof, can be included in the compositions and used in the methods described herein.

Various delivery vehicles can be used for the miRNA-3075/miRNA-3075-5p and/or for nucleic acid inhibitors of miR-434/miR-434-3p. In one embodiment, the delivery vehicle is a naturally occurring polymer, e.g., formed of materials including but not limited to albumin, collagen, fibrin, alginate, extracellular matrix (ECM), e.g., xenogeneic ECM, hyaluronan (hyaluronic acid), chitosan, gelatin, keratin, potato starch hydrolyzed for use in electrophoresis, or agar-agar (agarose). In one embodiment, the delivery vehicle comprises a hydrogel. In one embodiment, the composition comprises a naturally occurring polymer. For example, the miRNA or inhibitor thereof may be in nanoparticles or microparticles. Table 1 provides exemplary materials for delivery vehicles that are formed of naturally occurring polymers and materials for particles.

TABLE 1

| Particle class | Materials |
|---|---|
| Natural materials or | Chitosan |
| derivatives | Dextran |
| | Gelatine |
| | Albumin |
| | Alginates |
| | Liposomes |
| | Starch |
| Polymer carriers | Polylactic acid |
| | Poly(cyano)acrylates |
| | Polyethyleneimine |
| | Block copolymers |
| | Polycaprolactone |

An exemplary polycaprolactone is methoxy poly(ethylene glycol)/poly(epsilon caprolactone). An exemplary poly lactic acid is poly(D,L-lactic-co-glycolic)acid (PLGA).

Some examples of materials for particle formation include but are not limited to agar acrylic polymers, polyacrylic acid, poly acryl methacrylate, gelatin, poly(lactic acid), pectin(poly glycolic acid), cellulose derivatives, cellulose acetate phthalate, nitrate, ethyl cellulose, hydroxyl ethyl cellulose, hydroxypropylcellulose, hydroxyl propyl methyl cellulose, hydroxypropylmethylcellulose phthalate, methyl cellulose, sodium carboxymethylcellulose, poly(ortho esters), polyurethanes, poly(ethylene glycol), poly(ethylene vinyl acetate), polydimethylsiloxane, poly(vinyl acetate phthalate), polyvinyl alcohol, polyvinyl pyrrolidone, and shellac. Soluble starch and its derivatives for particle preparation include amylodextrin, amylopectin and carboxy methyl starch.

In one embodiment, the polymers in the nanoparticles or microparticles are biodegradable. Examples of biodegradable polymers useful in particles preparation include synthetic polymers, e.g., polyesters, poly(ortho esters), polyanhydrides, or polyphosphazenes; natural polymers including proteins (e.g., collagen, gelatin, and albumin), or polysaccharides (e.g., starch, dextran, hyaluronic acid, and chitosan). For instance, a biocompatible polymer includes poly (lactic) acid (PLA), poly (glycolic acid) (PLGA). Natural polymers that may be employed in particles (or as the delivery vehicle) include but are not limited to albumin, chitin, starch, collagen, chitosan, dextrin, gelatin, hyaluronic acid, dextran, fibrinogen, alginic acid, casein, fibrin, and polyanhydrides.

In one embodiment, the delivery vehicle is a hydrogel. Hydrogels can be classified as those with chemically crosslinked networks having permanent junctions or those with physical networks having transient junctions arising from polymer chain entanglements or physical interactions, e.g., ionic interactions, hydrogen bonds or hydrophobic interactions. Natural materials useful in hydrogels include natural polymers, which are biocompatible. biodegradable, support cellular activities, and include proteins like fibrin, collagen and gelatin, and polysaccharides like starch, alginate and agarose.

In one embodiment, a non-viral delivery vehicle comprises inorganic nanoparticles, e.g., calcium phosphate or silica particles; polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

Viral vectors for delivery are also envisioned, e.g., retrovirus lentivirus, adenovirus, herpesvirus or adeno-associated virus vectors.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines. A subset composed of these carbohydrates and four ethyleneamines in the polymer repeat units yielded exceptional delivery efficiency.

In one embodiment, the delivery vehicle comprises polyethyleneimine (PEI), polyamidoamine (PAMAM), PEI-PEG, PEI-PEG-mannose, dextran-PEI, OVA conjugate, PLGA microparticles, or PLGA microparticles coated with PAMAM.

In one embodiment, the delivery vehicle comprises a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed. In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C16:1, Cis:1 and C20:1) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, no delivery vehicle is employed, e.g., naked chemically modified RNA (cmRNA) is employed alone or with a scaffold.

In one embodiment, physical methods including but not limited to electroporation, sonoporation, magnetoporation, ultrasound or needle injection may be employed to introduce naked miRNA, complexes of miRNA and a delivery vehicle or miRNA encapsulated in particles.

### Inhibitory Nucleic Acids

The expression of miR-434, miR-3p, or any combination thereof can be inhibited, for example by use of an inhibitory nucleic acid that specifically recognizes a nucleic acid that encodes the mir-434 or the mir-434-3p.

An inhibitory nucleic acid can have at least one segment that will hybridize to a mir-434 and/or a miR-3p nucleic acid under intracellular or stringent conditions. The inhibitory nucleic acid can reduce expression of a nucleic acid encoding mir-434 or a mir-434-3p. A nucleic acid may hybridize to a genomic DNA, a messenger RNA, or a combination thereof. An inhibitory nucleic acid may be incorporated into a plasmid vector or viral DNA. It may be single stranded or double stranded, circular or linear.

An inhibitory nucleic acid is a polymer of ribose nucleotides or deoxyribose nucleotides having more than 13 nucleotides in length. An inhibitory nucleic acid may include naturally occurring nucleotides; synthetic, modified, or pseudo-nucleotides such as phosphorothiolates; as well as nucleotides having a detectable label such as P³², biotin or digoxigenin. An inhibitory nucleic acid can reduce the expression and/or activity of a mir-434 nucleic acid and/or a mir-434-3p nucleic acid. Such an inhibitory nucleic acid may be completely complementary to a segment of an endogenous mir-434 nucleic acid (e.g., an RNA) or an endogenous mir-434-3p nucleic acid (e.g., an RNA). Alternatively, some variability is permitted in the inhibitory nucleic acid sequences relative to mir-434 or mir-434-3p sequences. An inhibitory nucleic acid can hybridize to a mir-434 nucleic acid or a mir-434-3p nucleic acid under intracellular conditions or under stringent hybridization conditions and is sufficiently complementary to inhibit expression of the endogenous mir-434 nucleic acid or the endogenous mir-434-3p nucleic acid. Intracellular conditions refer to conditions such as temperature, pH and salt concentrations typically found inside a cell, e.g. an animal or mammalian cell. One example of such an animal or mammalian cell is a myeloid progenitor cell. Another example of such an animal or mammalian cell is a more differentiated cell derived from a myeloid progenitor cell. Generally, stringent hybridization conditions are selected to be about 5° C. lower than the thermal melting point (Tm) for the specific sequence at a defined ionic strength and pH. However, stringent conditions encompass temperatures in the range of about 1° C. to about 20° C. lower than the thermal melting point of the selected sequence, depending upon the desired degree of stringency as otherwise qualified herein. Inhibitory oligonucleotides that comprise, for example, 2, 3, 4, or 5 or more stretches of contiguous nucleotides that are precisely complementary to a mir-434 coding sequence or a mir-434-3p coding sequence, each separated by a stretch of contiguous nucleotides that are not complementary to adjacent coding sequences, can inhibit the function of a mir-434 and/or mir-434-3p. In general, each stretch of contiguous nucleotides is at least 4, 5, 6, 7, or 8 or more nucleotides in length. Non-complementary intervening sequences may be 1, 2, 3, or 4 nucleotides in length. One skilled in the art can easily use the calculated melting point of an inhibitory nucleic acid hybridized to a sense nucleic acid to estimate the degree of mismatching that will be tolerated for inhibiting expression of a particular target nucleic acid. Inhibitory nucleic acids of the invention include, for example, a short hairpin RNA, a small interfering RNA, a ribozyme or an antisense nucleic acid molecule.

The inhibitory nucleic acid molecule may be single or double stranded (e.g. a small interfering RNA (siRNA)) and may function in an enzyme-dependent manner or by steric blocking. Inhibitory nucleic acid molecules that function in an enzyme-dependent manner include forms dependent on RNase H activity to degrade target mRNA. These include single-stranded DNA, RNA, and phosphorothioate molecules, as well as the double-stranded RNAi/siRNA system that involves target mRNA recognition through sense-antisense strand pairing followed by degradation of the target mRNA by the RNA-induced silencing complex. Steric blocking inhibitory nucleic acids, which are RNase-H independent, interfere with gene expression or other mRNA-dependent cellular processes by binding to a target mRNA and getting in the way of other processes. Steric blocking inhibitory nucleic acids include 2′-0 alkyl (usually in chimeras with RNase-H dependent antisense), peptide nucleic acid (PNA), locked nucleic acid (LNA) and morpholino antisense.

Small interfering RNAs, for example, may be used to specifically reduce binding of mir-434 and/or mir-434-3p to target mRNAs. See, for example, website at invitrogen.com/site/us/en/home/Products-and-Services/Applications/mai.html. Once incorporated into an RNA-induced silencing complex, siRNA mediate cleavage of the homologous endogenous mRNA transcript by guiding the complex to the homologous mRNA transcript, which is then cleaved by the complex. The siRNA may be homologous and/or complementary to any region of the mir-434 and/or mir-434-3p. The region of homology may be 30 nucleotides or less in length, preferable less than 25 nucleotides, and more preferably about 21 to 23 nucleotides in length. SiRNA is typically double stranded and may have two-nucleotide 3′ overhangs, for example, 3′ overhanging UU dinucleotides. Methods for designing siRNAs are known to those skilled in the art. See, for example, Elbashir et al. Nature 411: 494-498 (2001); Harborth et al. Antisense Nucleic Acid Drug Dev. 13: 83-106 (2003).

The pSuppressorNeo vector for expressing hairpin siRNA, commercially available from IMGENEX (San Diego, California), can be used to generate siRNA for inhibiting expression of mir-434 and/or mir-434-3p. The construction of the siRNA expression plasmid involves the selection of the target region of the mRNA, which can be a trial-and-error process.

SiRNAs may be chemically synthesized, created by in vitro transcription, or expressed from an siRNA expression vector or a PCR expression cassette. See, e.g., website at invitrogen.com/site/us/en/home/Products-and-Services/Applications/mai.html. When an siRNA is expressed from an expression vector or a PCR expression cassette, the insert encoding the siRNA may be expressed as an RNA transcript that can in some cases folds into an siRNA hairpin. Thus, the RNA transcript may include a sense siRNA sequence that is linked to its reverse complementary antisense siRNA sequence by a spacer sequence that forms the loop of the hairpin as well as a string of U's at the 3′ end. The loop of the hairpin may be of any appropriate lengths, for example, 3 to 30 nucleotides in length, preferably, 3 to 23 nucleotides in length, and may be of various nucleotide sequences including, AUG, CCC, UUCG, CCACC, CTCGAG, AAGCUU, CCACACC and UUCAAGAGA (SEQ ID NO: 15). SiRNAs also may be produced in vivo by cleavage of double-stranded RNA introduced directly or via a transgene or virus. Amplification by an RNA-dependent RNA polymerase may occur in some organisms.

An inhibitory nucleic acid such as a short hairpin RNA siRNA or an antisense oligonucleotide may be prepared using methods such as by expression from an expression vector or expression cassette that includes the sequence of the inhibitory nucleic acid. Alternatively, it may be prepared by chemical synthesis using naturally occurring nucleotides, modified nucleotides or any combinations thereof. In some embodiments, the inhibitory nucleic acids are made from modified nucleotides or non-phosphodiester bonds, for example, that are designed to increase biological stability of the inhibitory nucleic acid or to increase intracellular stability of the duplex formed between the inhibitory nucleic acid and the target mir-434 or the mir-434-3p.

### Formulations and Dosages

The miRNA-3075-5p can be formulated as pharmaceutical compositions and administered to a mammalian host, such as a human patient in a variety of forms adapted to the chosen route of administration, e.g., orally or parenterally, by intravenous, intramuscular, topical, local, or subcutaneous routes. In one embodiment, the composition having isolated polypeptide or peptide is administered to a site of bone loss or cartilage damage or is administered prophylactically.

In one embodiment, the miRNA-3075-5p may be administered by infusion or injection. Solutions of the miRNA or its salts, can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in complexes, liposomes, nanoparticles or microparticles. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In some cases, it may be preferable to include isotonic agents, for example, sugars, buffers or sodium chloride. Prolonged absorption of the injectable compositions can be brought about by the use in the compositions of agents delaying absorption, for example, microparticles, or aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the miRNA-3075-5p can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the miRNA in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the miRNAs for use alone or with other agents will vary with the route of administration, the nature of the condition being treated and the age and condition of the patient and will be ultimately at the discretion of the attendant physician or clinician.

The miRNA may be conveniently administered in unit dosage form; for example, containing 5 to 1000 mg, conveniently 10 to 750 mg, or conveniently 50 to 500 mg of active ingredient per unit dosage form.

In general, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

Summary of Experimental Results The experimental work described herein illustrates the effect of hepatocytes on systemic insulin sensitivity during the early development of obesity and after full establishment of the obese condition in HFD-fed mice. The results show that Exos derived from hepatocytes during the early and late stages of obesity provide divergent signals which regulate the state of systemic insulin sensitivity. Exos secreted from hepatocytes during the early stage of obesity (4 weeks of the high fat diet (HFD)) directly mediate enhanced insulin sensitivity in adipocytes, skeletal muscle cells (L6 myocytes), and primary hepatocytes. These 4 week HFD Exos also induced improved glucose tolerance and systemic insulin sensitivity when administered to insulin-resistant 16 week HFD/obese wild type mice. The results described herein also show that the miRNA cargo of these Exos is responsible for these beneficial effects and that miR-3075 is a specific insulin-sensitizing miRNA that is highly expressed in 4 week HFD hepatocytes and their secreted Exos.

Thus, miR-3075, and Exos containing miR-3075, represent a compensatory response to the early stages of caloric excess to protect a subject from insulin resistance caused by chronic obesity. As a compensatory response, such regulation of Exos secretion and miRNA expression is similar to classical endocrine signaling.

After the development of established chronic HFD-induced obesity, hepatocyte Exos no longer contain high levels of miR-3075 and do not mediate increased insulin sensitivity. In fact, hepatocyte-derived Exos from chronically obese mice promote insulin resistance by activating proinflammatory responses in macrophages. Thus, early in the course of obesity, hepatocytes produce insulin-sensitizing Exos, but when obesity is fully established, this compensatory effect is lost, and hepatocyte Exos from chronic HFD mice promote insulin resistance. A high fat diet leads to the gradual development of obesity in mice which typically plateaus at 12-16 weeks of HFD feeding.

Mice fed a HFD for 4 weeks were used as a model for early onset obesity while mice fed a HFD for 16 weeks were used as an example of chronic, established obesity. Glucose tolerance and insulin sensitivity are severely compromised in chronically obese mice compared to lean controls, while glucose tolerance and insulin sensitivity in 4 weeks HFD are intermediate.

The inventors noted that infusions of lipid emulsions, which are often used as a model of high fat intake, cause insulin resistance within 1-2 hours, and hypothesized that there might be some compensatory response in early onset obesity mitigating the full development of insulin resistance and hyperglycemia. The experiments described herein show that primary hepatocytes isolated from 4 week HFD mice exhibit normal insulin sensitivity compared to hepatocytes from lean mice, whereas hepatocytes from mice fed a HFD for 16 weeks mice are highly insulin resistant. These results indicate that in early onset obesity, hepatocytes do not manifest cell-autonomous insulin resistance.

The inventors postulated that hepatocyte-derived Exos could cause biological effects contributing to the observe metabolic phenotypes. To assess this, Exos were harvested from primary hepatocytes derived from lean mice, 4 week HFD mice, and 16 week HFD mice. In early onset obesity, the 4 week HFD hepatocyte Exos have a beneficial effect on insulin sensitivity. This was demonstrated by treating 3T3-L1 adipocytes, L6 myocytes, and insulin-resistant primary mouse hepatocytes with these Exos preparations and showing that this directly leads to enhanced insulin signaling in all 3 cell types. Furthermore, treatment of chronically obese 16 week HFD/obese animals with hepatocyte Exos derived from 4 week HFD mice led to improved glucose tolerance with less insulin resistance. These findings suggest that in early onset obesity hepatocytes secrete Exos which act to partially maintain insulin sensitivity, consistent with a compensatory response to prevent the severe metabolic dysregulation which eventually appears in the chronically obese mice.

Two hepatocyte-specific knockout models were generated as described herein, each designed to eliminate or greatly reduce loading of endogenous miRNAs into secreted Exos. Hepatocyte-specific knockout of Rab27 led to decreased hepatocyte Exos secretion with a substantially greater decrease in Exos miRNA content. The YBX1 knockout model led to essentially complete depletion of Exos miRNAs. In both cases, depletion of hepatocyte Exos miRNAs led to exaggerated glucose intolerance and reduced insulin sensitivity in the knockout mice, supporting a conclusion that hepatocyte Exos miRNAs in early onset obesity function to partially counteract the adverse metabolic effects of HFD and obesity. Consistent with such a conclusion, in vitro studies in primary hepatocytes showed that insulin sensitivity is normal after four weeks of HFD, whereas the hepatocytes become markedly insulin resistant after 16 weeks of HFD. Interestingly, while hepatocyte insulin sensitivity remains normal in the 4-week HFD wild type mice, the cells become insulin resistant when prepared from 4-week HFD Rab27-KO mice or YBX1-KO mice.

To further explore the observed miRNA-induced phenotypes, miRNA sequencing was performed on primary hepatocyte-derived Exos from the different wild type mouse cohorts. It was found that miR-3075 is highly expressed in 4 week HFD hepatocyte Exos compared to hepatocytes from lean mice, and that expression of miR-3075 returns to normal levels in the 16 week HFD hepatocyte Exos.

When miR-3075 levels were measured in adipose tissue and skeletal muscle in 4 week HFD hepatocyte-specific Rab27 knockout mice, the levels of miR-3075 were not elevated and were comparable to values observed in chow-fed lean mice or chronic HFD obese mice.

The results described herein shows that hepatocyte-derived miR-3075 leads to systemic insulin sensitization, and this concept was well supported by in vitro and in vivo studies showing that a miR-3075 nucleic acid is sufficient to enhance insulin sensitivity and glucose tolerance.

To further fortify such conclusions, an antisense approach was used to specifically inhibit miR-3075 using specific antagomirs directed against miR-3075 that interfered with the ability of the miR-3075 to bind to its mRNA targets. A miR-3075 antagomir that was used with a miR-3075 mimic to cotreat insulin target cells completely blocked the effects of the miR-3075 mimic to enhance insulin sensitivity. Importantly, the miR-3075 antagomir completely, or essentially completely, blocked the effects of 4 week HFD Exos to improve insulin signaling, indicating that miR-3075 is the major miRNA within the hepatocyte exosomes causing the increase in insulin sensitivity. Importantly, when 4 week HFD mice were treated in vivo with the miR-3075 antagomir, glucose tolerance deteriorated and insulin sensitivity decreased. Such results indicate that endogenous miR-3075 is attenuating insulin resistance during early onset obese situation, by contributing to improved insulin action.

In vivo treatment of chow-fed mice with the antagomir had little or no effects, likely because endogenous levels of miR-3075 are very low in the chow-fed condition and only rise in the early onset obese model (then decline back down to low levels during chronic obesity). Although it is not possible to strictly compare in vivo measurements of glucose or insulin tolerance quantitatively, the effects of the antagomir to impair glucose tolerance in 4 week HFD mice are similar in magnitude to the effects of the miR-3075 mimic to improve glucose tolerance in chronic obese mice. Such observation indicate that miR-3075 is either the unique or dominant miRNA within 4 week HFD hepatocyte Exos causing the beneficial phenotypic effects. Taken together, these studies show that miR-448 3075 is an insulin-sensitizing miRNA contained in hepatocyte Exos from 4 weeks HFD mice.

miRNAs exert their biologic effects by binding through their seed sequence to complementary nucleotides in the 3′ region of target mRNAs leading to translational arrest or degradation (Bartel, 2009). Using a bioinformatics approach, several theoretical target mRNAs were identified and it was demonstrated that one of these, FA2H, was highly repressed in adipose tissue, muscle, and liver by in vivo treatment with either the 4 week HFD hepatocyte Exos or the miR-3075 mimic. These results indicate that the mRNA for FA2H is a target for miR-3075, and this was directly demonstrated by showing suppression of a luciferase reporter construct containing the FA2H 3⁺ UTR. Using a complementary approach to support these concepts, FA2H was depleted in three main insulin target cell types by treatment with a siRNA directed against FA2H. Suppression of FA2H led to enhanced in vitro insulin signaling, comparable to the effects induced by treatment with 4 week HFD Exos or the miR-3075 mimic. In aggregate, these results show that use of miR-3075 nucleic acids can have therapeutic value in insulin-resistant states.

By 16 week of HFD, obesity is chronic and well established in mice and they become markedly glucose intolerant and insulin resistant. Interestingly, during the chronic obese condition, hepatocytes no longer secrete insulin-sensitizing Exos. Such a compensatory response by 4 week HFD hepatocyte Exos is lost. In the chronic obese state, hepatocytes now secrete Exos which promote insulin resistance by augmenting an adipose tissue macrophage (ATM) proinflammatory state. Thus, 16 week HFD Exos cause glucose intolerance and insulin resistance when administered to lean chow mice and specifically promote the M1-like polarization state of ATMs and inhibit M2-like ATM polarization. Because these 16 week HFD Exos do not directly impair insulin signaling in vitro within adipocytes, myocytes, and primary hepatocytes, the mechanism whereby these Exos promote insulin resistance is likely by stimulating the macrophage proinflammatory state. The miRNA sequencing data on Exos from these various groups are fully consistent with this conclusion. Thus, the insulin-sensitizing miR-3075, which is highly expressed in 4 week HFD hepatocyte Exos, shows a low expression level in 16 week HFD hepatocyte Exos. In addition, Exos obtained from chronic obese mice express high levels of miR-434-3p, and the experiments described herein show that this miRNA can directly promote macrophage polarization towards the M1-like state both in vitro and in vivo. This conclusion was further validated by experiments in which the ability of hepatocytes to produce miRNAs was deleted by using hepatocyte-specific Rab27 or YBX1 knockout mice. Deletion of hepatocyte miRNAs in the chronic obese state led to markedly improved glucose tolerance and insulin sensitivity, consistent with the conclusion that these hepatocytes secrete Exos that promote insulin resistance.

In summary, it was shown that during the course of obesity hepatocyte-derived Exos have important effects to modulate systemic glucose tolerance and insulin sensitivity. Early in the onset of obesity, hepatocytes secrete Exos that promote an insulin-sensitive state, presumably as a compensatory response to the caloric excess which leads to the opposite effect by causing insulin resistance. These beneficial properties of 4 week HFD Exos are mediated through their miRNA cargo. MiR-3075 was identified as the dominant insulin-sensitizing molecule within these Exos. Treatment with a miR-3075 nucleic acids led directly to improved insulin signaling in vitro, and when given in vivo, caused improved glucose tolerance and heightened insulin sensitivity. As the obesity process progresses to the chronic state, these insulin-sensitizing Exos are no longer produced, and, instead, hepatocytes now make Exos containing miRNAs (e.g., miR-434-3p) that directly promote insulin resistance and glucose intolerance by stimulating proinflammatory polarization of ATMs. Therefore, the miR-3075 can be used as an insulin-sensitizing therapeutic. In addition, an antagomir directed against miR-434-3p can also have beneficial effects.

The invention will be described by the following non-limiting examples.

### EXAMPLE

Insulin resistance is a key pathophysiologic feature of obesity and Type 2 Diabetes Mellitus. In the chronic obese state, hepatocytes become insulin resistant and exert important effects on systemic metabolic homeostasis. Here we show that in early onset obesity (4 wks HFD), hepatocytes secrete exosomes (Exos) which enhance insulin sensitivity in vitro in 3T3-L1 adipocytes, L6 myocytes and primary hepatocytes. More importantly, these Exos also improve glucose tolerance and insulin sensitivity when administered in vivo to insulin resistant, chronically obese mice. These beneficial effects are due to the exosomal miRNA cargo, since Exos in which all miRNAs are depleted do not cause these insulin sensitizing effects. miR-3075 is highly enriched in these early stage 4 week HFD hepatocyte Exos and studies utilizing an miR-3075 mimic or miR-3075 antagomir show that this miRNA is largely responsible for the insulin sensitizing effects of 4 week HFD hepatocyte Exos. FA2H is a direct target of miR-3075 and siRNA depletion of FA2H in adipocytes, myocytes, and primary hepatocytes leads to increased insulin sensitivity. In contrast to early stage HFD, in chronic, established obesity (16-18 weeks HFD), hepatocyte Exos promote a state of insulin resistance. While these chronic obese hepatocyte Exos do not directly cause impaired insulin signaling in vitro, they promote proinflammatory activation in macrophages. This indicates that the insulin resistance induced by chronic obese hepatocyte Exos is secondary to increased tissue inflammation. Taken together, these studies show that in early onset obesity, hepatocytes produce Exos which express high levels of the insulin sensitizing miR-3075. In contrast, when chronic obesity is established, this compensatory effect is lost and hepatocyte-derived Exos from 3 chronic obese mice promote insulin resistance. miR-3075 is a potential new insulin sensitizing molecule which could have therapeutic potential.

### Example 1: Materials and Methods

Mice. C57BL/6 (B6) mice were fed a high-fat diet (60% fat calories, 20% protein calories, and 20% carbohydrate calories; Research Diets) or a normal chow diet ad libitum. In most assays, the mice were fed with HFD for 12-16 weeks. B6 WT, Albumin-Cre and Rosa26-floxed STOP-Cas9 knockin mice were purchased from the Jackson Laboratory (Table 2). To generate hepatocyte-specific Cas9 transgenic mice, Rosa26-floxed STOP-Cas9 knockin mice were bred with Albumin-Cre mice. All mice were maintained on a 12/12-hour light-dark cycle. In the experiments using hepatocyte-specific Cas9 transgenic mice, Rosa26-floxed STOP-Cas9 knockin mice were used as controls. All mice used in this study were male and maintained on at 22° C. in a 12/12-hour light-dark cycle in a specific pathogen-free facility and given free access to food and water. All animal procedures were done in accordance with University of California, San Diego Research Guidelines for the Care and Use of Laboratory Animals and all animals were randomly assigned to cohorts when used.

Differentiation of adipocytes. To generate adipocytes, 3T3-L1 cells were differentiated in the induction medium (DMEM/F12 medium containing 4.5 g/L glucose, 10% FBS, penicillin-streptomycin, and glutamine and then induced with a differentiation cocktail consisting of 0.5 mM 3-isobutyl-1-methylxanthine, 1 μM dexamethasone, 10 μg/mL insulin, 0.2 mM indomethacin, and 1 μM rosiglitazone in DMEM supplemented with 10% FBS, PS, and glutamine) for 7 days.

Isolation of primary hepatocytes. Primary hepatocytes were isolated as described previously (Ying et al., 2017; Li et al., 2016). Briefly, mice were is infused with a calcium-free HEPES-phosphate buffer A (Calcium and magnesium-free PBS containing 0.2 μM EGTA, 10 mM HEPES, 1 mM glucose, and 0.2% BSA, pH 7.4) via the vena cava for 3-5 minutes. After the color of the liver changed to a beige or light brown color, collagenase-containing buffer B (PBS with 1 mM magnesium and 1 mM calcium, 0.2% BSA, and 30 mM HEPES, 0.5 mg/ml collagenase D) was perfused into liver (Table 2). After the appearance of cracking on the surface of liver, perfusion was stopped immediately and the liver was excised into ice-cold buffer A. Cells from digested livers were teased out, suspended in Buffer A, filtered through 100 m cell strainer, and centrifuged at 60×g for 6 minutes at 4° C. The pellet was washed with Buffer B (no collagenase) twice and then mixed with Percoll (adjusted to physiological ionic strength with 10×PBS) to a final concentration of 36% and centrifuged at 100×g for 10 minutes, 4° C. After removing the supernatant, the hepatocyte pellet was washed once with Buffer B (without collagenase) and then cultured in Williams Medium E containing 10% FBS on collagen-coated plates (GIBCO, Life Technologies) and antibiotics. After overnight incubation (16 hours), culture medium was refreshed.

Glucose tolerance and insulin tolerance tests. For glucose tolerance tests, mice received one dose of dextrose (1 g/kg body weight) via intraperitoneal (i.p.) injection after 16 hours of fasting. For insulin tolerance tests, mice were fasted for 6 hour and then i.p. injected with insulin (0.35 or 0.175 units/kg body weight for HFD or NCD mice, respectively).

Hyperinsulinemic-euglycemic clamp studies. To perform hyperinsulinemic-euglycemic clamp assays, mice were surgically implanted with jugular vein cannulation. Three to five days after recovery, 6-hour fasted mice were infused with D-[3-³H] glucose (Perkin Elmer) for 90 minutes. After tracer equilibration, blood sampling occurred, then glucose (50% dextrose) and tracer (5 μCi/h) plus insulin (8 mU/kg/min) were infused into the jugular vein. Blood samples were measured from the tail vein at 10 minutes intervals. The steady-state conditions (120 mg/dl±10 mg/dl) were confirmed at the end of the clamp by maintaining glucose infusion and plasma glucose concentration for a minimum of 20 minutes. Blood samples at time point=−10, 0 (basal), 110, and 120 (end of experiment) minutes were collected to determine glucose-specific activity, as well as free fatty acid and insulin concentration. Tracer-determined rates were quantified by using the Steele equation for steady-state conditions. At steady state, the rate of glucose disappearance (GDR) is equal to the sum of the rate of endogenous glucose productions (HGP) plus the exogenous GIR. The IS-GDR is equal to the total GDR minus the basal glucose turnover rate.

In vivo insulin-stimulated AKT phosphorylation assay. Tissue insulin action was evaluated by measuring insulin-stimulated AKT phosphorylation in liver, skeletal muscle, and epididymal white adipose (eWAT). Briefly, after 16 hours fasting, mice were anesthetized and parts of these insulin target tissues were collected to measure basal level of AKT phosphorylation. After a dose of insulin (0.35 or 0.175 U/kg body weight for HFD or NCD mice, respectively) injected via vena cava, parts of the liver, skeletal muscle, and eWAT were collected at 3 minutes, 7 minutes, and 10 minutes, respectively. The phosphorylation (Ser473) of AKT and Pan-AKT abundance were measured using Western Blots analysis.

Exos purification and characterization. The Exos from primary hepatocyte culture medium were prepared as previously described (Ying et al. 2017). After 24 hours culture, debris and dead cells in the medium were removed by centrifugation at 1,000×g for 10 minutes and then filtrated through a 0.2 μm filter. The medium was then subjected to ultracentrifugation at 100,000×g for 4 hours at 4° C. After wash with PBS (100,000×g for 20 minutes), the Exos-containing pellet was resuspended in 1 ml PBS and passed through a 0.2 m filter to remove large particles. The Exos particle size and concentration were measured by NanoSight analysis (Malvern Instruments). To monitor Exos trafficking, Exos were labeled with PKH26 fluorescent dye using the PKH26 fluorescent cell linker kit (Sigma-Aldrich). After PKH26 staining, the Exos were washed with PBS and collected by ultracentrifugation (100,000×g for 2 hours) at 4° C. Finally, PKH26 labeled Exos were resuspended in PBS.

In vivo Exos trafficking assays. PKH26-labeled hepatocyte Exos (1×10⁹ EVs per mouse) were delivered to HFD recipient mice through injection into tail vein. After 16 hours Exos injection, parts of liver, skeletal muscle, and eWAT were collected for detecting the appearance of PKH26 red fluorescence.

In vivo and in vitro Exos treatment. For in vitro assays, 1×10⁸ Exos on the basis of NanoSight analysis were added to 0.5×10⁶ cells for 36 hours. For in vivo treatment, recipient mice were intravenously injected 5×10⁹ EVs twice per week.

Differentiation of bone marrow-derived macrophages. Bone marrow-derived macrophages (BMDMs) were prepared as previously described (Ying et al., 2013). BMDMs were treated with IL4 (20 ng/mL) and IL13 (10 ng/mL) or lipopolysaccharide (LPS, 100 ng/mL) to stimulate M2 or M1 polarization, respectively. M1 macrophages are classically activated, for example by IFN-γ or lipopolysaccharide (LPS), and produce proinflammatory cytokines, phagocytize microbes, and initiate an immune response. M1 macrophages produce nitric oxide (NO) or reactive oxygen intermediates (ROI) to protect against bacteria and viruses. M2 macrophages respond to stimuli such as IL-4 or IL-13 and are producers of anti-inflammatory cytokines.

miRNA mimic or antagomir transfection or in vivo treatment. miRNA mimic or antagomir was transfected into recipient cells with the lipofectamine RNAiMAX reagent (ThermoFisher Scientific). MiR-3075-5p mimics and miR-434-3p mimics are now made to order and available from ThermoFisher Scientific as catalog no. 4464066. After 24 hours, the transfection efficiencies were validated by qPCR analysis. MiR-3075-5p inhibitors/antagomirs are now made to order and available from ThermoFisher Scientific as catalog nos. 4464084 and AM17000. MiR-434-3p inhibitors/antagomirs are now made to order and available from ThermoFisher Scientific as catalog nos. 4464084 and AM17000.

For the in vivo delivery, the miR-3075 mimic or antagomir was encapsulated with Invivofectamine and then administrated into either HFD-fed (starting at 1 week HFD feeding) or NCD recipient WT mice (10 nmol mimic per mouse, twice per week) through tail vein injection.

Immunofluorescence staining. Pieces of liver, skeletal muscle and eWAT of HFD mice were snap frozen in optimum cutting temperature (O.C.T., Fisher Healthcare) with dry ice. Six μm cryo-sections of tissue sections were cut and fixed with pre-cold acetone for 20 minutes. Immunostaining was performed as previously described. Slides were blocked with 5% normal donkey serum for 60 minutes at RT. After washing, nuclei were stained with DAPI (4′,6-Diamidino-2-28 phenylindole dihydrochloride) for 10 minutes at room temperature. Mounting media and cover slips were then added to slides for imaging. Images were acquired on a Keyence Fluorescent Microscope and were processed with ImageJ (NIH, Bethesda, MD).

Quantitative Reverse Transcriptase-polymerase Chain Reaction (RT-PCR) Analysis. Total RNA was extracted from islet macrophages using the RNA extraction protocol according to the manufacturer's instructions (Zymo Research). cDNA was synthesized using SuperScript III and random hexamers. qPCR was carried out in 10 μl 703 reactions using iTaq SYBR Green supermix on a StepOnePlus Real-Time PCR Systems (ThermoFisher Scientific). For miRNA RT-PCR, cDNA was synthesized using TaqMan™ 705 microRNA reverse transcription kit and miRNA primers (5×) (Table 2). qPCR was performed using TaqMan™ universal master mix II and miRNA primers (20×) in 10 μl reactions on a StepOnePlus Real-Time PCR Systems (ThermoFisher Scientific). The data presented correspond to the mean of 2-A^{C}t from at least three independent experiments after being normalized to β-actin or U6.

Glucose uptake assay. After 12 hour serum starvation, cells were stimulated with 100 nM insulin for 30 minutes in KRH buffer (137 nM NaCl, 4.8 mM KCl, 1.2 mM KH₂PO₄, 1.2 mM 712 MgSO₄, 2.5 mM CaCl₂), 0.2% BSA, 16 mM HEPES) at 37° C. Then ³H-2-deoxy-D-glucose 713 (³H-2-DOG, 0.1 mM, 0.4 μCi/ml) was supplemented to cells. After 10 minutes incubation at 714 37° C., cells were washed with ice-cold PBS twice. NaOH (1 N) was then added and incubated for 20 minutes to efficiently dissolve cells. An aliquot was used for protein concentration measurement. After neutralizing NaOH by adding HCl (1 N), the extracts were transferred to scintillation vials, and scintillation fluid was added and the radioactivity was counted. Results were normalized with protein concentration of cell lysates.

Glucose output assay. After 12 hours serum starvation, primary hepatocytes were washed twice and then exposed to glucose-free buffer (10 mM HEPES, 4 mM KCl, 125 mM NaCl, 0.85 mM KH₂PO₄, 1.25 mM Na₂HPO₄, 1 mM CaCl₂), and 15 mM NaHCO₃) containing glucagon (200 ng/ml), insulin (10 nM), or a combination of glucagon and insulin for 4 hours, at 37° C. Glucose production was determined by the measurement of glucose in the media. The primary hepatocytes attached the culture plate were dissolved by adding NaOH (1 N) and protein content was determined. The glucose results were normalized with protein concentration of cell lysates.

Western blot analysis. Cells or tissues were homogenized in RIPA buffer supplemented with protease and phosphatase inhibitors. Equal amounts of cell lysate proteins (30 μg protein per lane for pAKT detection) from each biological replicate were subjected to western blotting. Using ChemiDoc XRS imaging system (BioRad), the protein bands on blots were detected with the SuperSignal West Pico Chemiluminescent Substrate. Protein bands were analyzed using Image Lab software (BioRad). Western blot data in figures and supplemental figures are all representative of more than three independent experiments. Antibody information is provided in Table 2.

Small RNA deep sequencing. Total RNA was isolated from either lean (n=4), 4 wks HFD 736 (n=4), or 16 wks HFD (n=4) hepatocyte-derived Exos using the Zymo Quick-RNA 737 MiniPrep kit. RNA purity was assessed by an Agilent 2100 Bioanalyzer. Sequencing libraries were prepared using the Illumina TruSeq Small RNA protocol with minor modification following the manufacturer's instructions with 12 cycles of PCR amplification after ligation of the 3′ and 5′ adapters. Samples were ligated to unique adaptors and subjected to PCR amplification. Libraries were then validated using an Agilent 2100 Bioanalyzer, normalized, and pooled for sequencing. RNA-seq libraries prepared from four biological replicates for each group as indicated were sequenced on an Illumina HiSeq 2500 using barcoded multiplexing.

miR-3075 target gene prediction and validation. miR-3075 target gene prediction was conducted with TargetScan Mouse 7.2 (www.targetscan.org). To validate that Fa2h is a genuine target gene of miR-3075, the luciferase reporter assay was carried out with 3′ untranslated regions of Fa2h containing potential WT miR-3075 binding sites inserted downstream from the Renilla luciferase gene. The reporter constructs were co-transfected with miR-3075 mimic or negative mimic control into HEK293 cells. After 24 hours co-transfection, the activities of Renilla luciferase were measured with the Dual-Glo 752 luciferase reporter system.

Quantification and statistical analysis. Blinding was performed whenever deemed to be appropriate and applicable. Sample description and identification was unavailable to the core personnel during data plotting and analyses. No samples or data were excluded from the study for statistical purposes. Each in vitro experiment was independently performed with duplicate or triplicate to ensure reproducibility. Group sizes of 5 mice or above were sufficient to reach a statistical power of at least 80%. Mice were assigned at random to treatment groups for all mouse studies. Tests used for statistical analyses are described in the figure legends. The sample numbers are mentioned in the figure legends. To assess whether the means of two groups are statistically different from each other, unpaired two-tailed Student's t-test was used for statistical analyses, all data passed the normality test using Prism8 software (GraphPad software v8.0; Prism, La Jolla, CA). P values of 0.05 or less were considered to be statistically significant. Degrees of significance were indicated in the figure legends. For the results of glucose and insulin tolerance tests, statistical comparisons between every two groups at each time point were performed with unpaired two-tailed Student's t-test.

TABLE 2
Reagents and resources

| | | |
|---|---|---|
| Reagent or Resource | Source | Identifier |

Antibodies

| | | |
|---|---|---|
| Anti-TSG101 | ThermoFisher | Cat# MA1-23296; |
| | Scientific | RRID: AB_2208088 |
| Anti-CD63 | ABclonal | Cat# A5271; |
| | | RRID: AB_2766092 |
| Anti-Syntenin 1 | ThermoFisher | Cat# PA5-28826; |
| | | RRID: AB_2546302 |
| Anti-FA2H | ABclonal | Cat# A13874; |
| | | RRID: AB_2760726 |
| Anti-HSP90 | Santa Cruz | Cat# sc-101494; |
| | | RRID: AB_1124018 |
| Anti-Rab27a | Cell signaling | Cat# 69295; |
| | technology | RRID: AB_2799759 |
| Anti-Rab27b | ABclonal | Cat# A8897; |
| | | RRID: AB_2771907 |
| Anti-YBX1 | ThermoFisher | Cat# PA5-80241; |
| | | RRID: AB_2747355 |
| Anti-GAPDH | Cell signaling | Cat# 2118S; |
| | technology | RRID: AB_561053 |
| Anti-HSP70 | ThermoFisher | Cat# MA5-31961; |
| | | RRID: AB_2809255 |
| Anti-phospho-P65 | Cell signaling | Cat# 3033S; |
| | technology | RRID: AB_331284 |
| Anti-P65 | Cell signaling | Cat# 8242P; |
| | technology | RRID: AB_10859369 |
| Anti-Phospho-AKT | Cell signaling | Cat# 9271S; |
| Ser473 | technology | RRID: AB_329825 |
| Anti-pan AKT | Cell signaling | Cat# 4691S; |
| | technology | RRID: AB_915783 |
| APC/Cyanine7 | BioLegend | Cat# 103115; |
| anti-CD45 | | RRID: AB_312980 |
| FITC anti-CD11b | BioLegend | Cat# 101205; |
| | | RRID: AB_312788 |
| PerCP/Cyanine5.5 | BioLegend | Cat# 141715; |
| ant-CD206 | | RRID: AB_2561991 |
| PE/Cy7 anti-F4/80 | BioLegend | Cat# 123113; |
| | | RRID: AB_893490 |
| Brilliant Violet 421 | BioLegend | Cat# 117329; |
| anti-CD11c | | RRID: AB_10897814 |

Chemicals, Peptides, and Recombinant Proteins

| | | |
|---|---|---|
| Live/Dead Fixable Aqua | ThermoFisher | Cat# L34966 |
| dead cell stain kit | | |
| Novolin R regular human | Novo-Nordisk | Cat# NDC 0169-1833-11 |
| insulin used in ITTs | | |
| Insulin used in glucose | Sigma-Aldrich | Cat# I9278 |
| uptake and hepatic glucose | | |
| production assays | | |
| Glucagon | Sigma-Aldrich | Cat# G2044-1MG |
| Dextrose | Hospira, Inc | Cat# 0409-6648-02 |
| 3H-glucose | Perkin Elmer | Cat# NET331C001MC |
| Collagenase II | Sigma-Aldrich | Cat# C2674 |
| Percoll | GE Healthcare | Cat# 17-0891-01 |
| | Life Sciences | |
| SuperSignal West Pico | ThermoFisher | Cat# 34077 |
| Chemiluminescent | Scientific | |
| Substrate | | |
| Halt Protease and | ThermoFisher | Cat# 78440 |
| Phosphatase Inhibitor | Scientific | |
| Cocktail | | |
| RIPA buffer (10x) | Cell Signaling | Cat# 9806 |
| | Technology | |
| PKH26 | Sigma-Aldrich | Cat# PKH26GL-1KT |
| 60% high fat diet | Research Diets | Cat# D12492 |
| Collagenase H | Roche | Cat# 11249002001 |
| RBC lysis buffer | eBioscience | Cat# 00-4333-57 |
| High-capacity cDNA | ThermoFisher | Cat# 4368813 |
| reverse transcription kit | Scientific | |
| iTaq SYBR Green supermix | Bio-Rad | Cat# 172-5125 |
| Exosome-depleted FBS | SBI | Cat# EXO-FBSHI-50A-1 |
| Invivofectamine | ThermoFisher | Cat# IVF3005 |
| | Scientific | |
| Lipofectamine RNAiMAX | ThermoFisher | Cat# 13778-075 |
| reagent | Scientific | |
| TaqMan ™ microRNA | ThermoFisher | Cat#4366597 |
| reverse transcription kit | Scientific | |
| TaqMan ™ universal master | ThermoFisher | Cat#4440040 |
| mix II | Scientific | |
| Recombinant murine IL4 | Peprotech | Cat# 214-14 |
| Recombinant murine IL13 | Peprotech | Cat# 210-13 |
| Lipopolysaccharide | Sigma-Aldrich | Cat# L2630 |

Commercial Assays

| | | |
|---|---|---|
| Quick-RNA MiniPrep kit | Zymo research | Cat#R1051 |
| Glucose colorimetric | Cayman | Cat# 10009582 |
| assay kit | | |
| Insulin ELISA kit | ALPCO | Cat# 80-INSHU-E01.1 |

Experimental Models: Cell Lines

| | | |
|---|---|---|
| 3T3-L1 cell line | ATCC | CRL-11506 |
| L6 cell line | ATCC | CRL-1458 |
| HEK293 cell line | ATCC | CRL-11268 |

Expermiental Models: Organisms/Strains

| | | |
|---|---|---|
| Mouse: WT C57BL6/J | Jackson | JAX: 000664 |
| | Laboratories | |
| Mouse: Albumin Cre | Jackson | JAX: 003574 |
| | Laboratories | |
| Mouse: Rosa26-floxed | Jackson | JAX: 026175 |
| STOP-Cas9 knockin | Laboratories | |

Oligonucleotides

| | | |
|---|---|---|
| miR-3075 RT-PCR | ThermoFisher | Assay ID: 461745_mat |
| primer | Scientific | |
| miR-434-3p RT-PCR | ThermoFisher | Assay ID: 002604 |
| primer | Scientific | |
| miR-3075 mimic | ThermoFisher | Assay ID: MC19722 |
| | Scientific | |
| miR-434-3p mimic | ThermoFisher | Assay ID: MC12870 |
| | Scientific | |
| HPLC-purified miR-3075 | Horizon | Cat# IH-311489-01 |
| antagomir (for in vivo | Discovery | |
| treatment) | | |
| miR-434-3p antagomir | Horizon | Cat# 310625-08-0002 |
| | Discovery | |
| miRNA mimic | ThermoFisher | Cat# 4464058 |
| negative control | Scientific | |

### Example 2: Hep Atocyte-Derived Exosomal miRNAs Enhance Insulin Sensitivity in Early Stage Obesity

Extracellular vesicles (EVs) play a role in transporting extracellular miRNAs between different cell types. EVs are heterogeneous membranous nanoparticles released from most cell types consisting of smaller particles termed Exos, larger microvesicles, and even larger apoptotic bodies. In these studies, a previously described isolation method was used that yields a highly enriched and well characterized population of Exos from cell culture media and, therefore, the term Exos is used herein (Ying et al., 2017; Ying et al., 2021).

To assess the effect of high fat diet (HFD) induced-obesity on insulin sensitivity, hepatocyte-derived Exos were collected from chow-fed lean animals, from mice fed the HFD for 4 week with early stage obesity (4 week HFD Exos), or from chronically obese mice fed the HFD for 16 week (16 week HFD Exos).

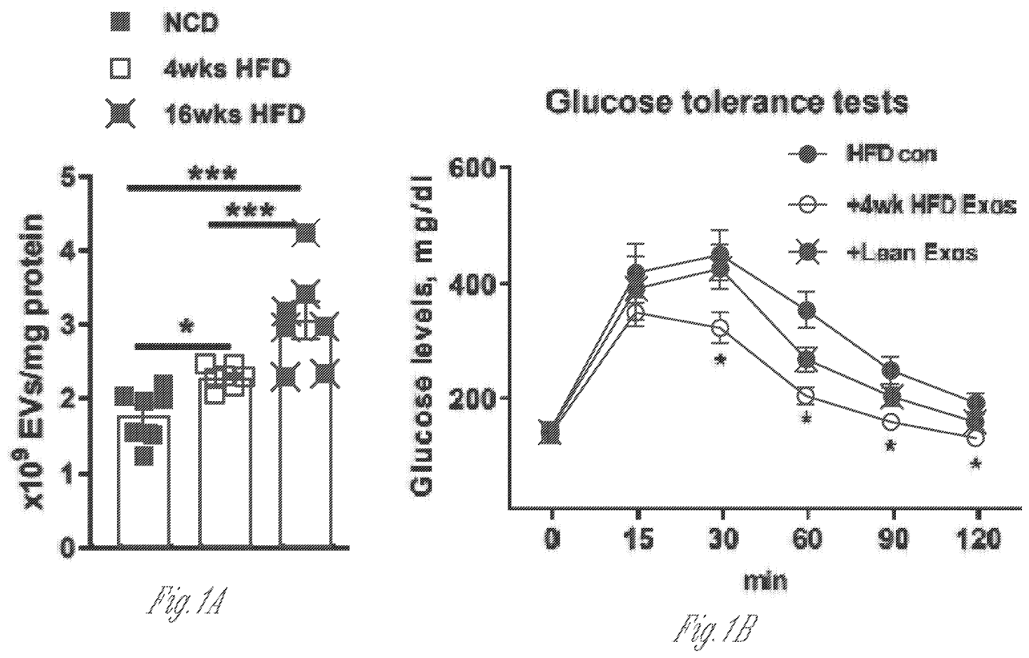

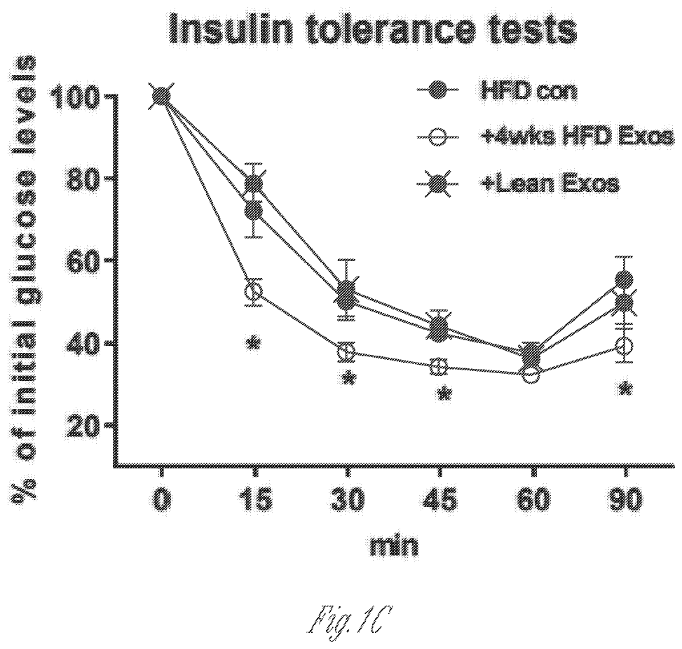

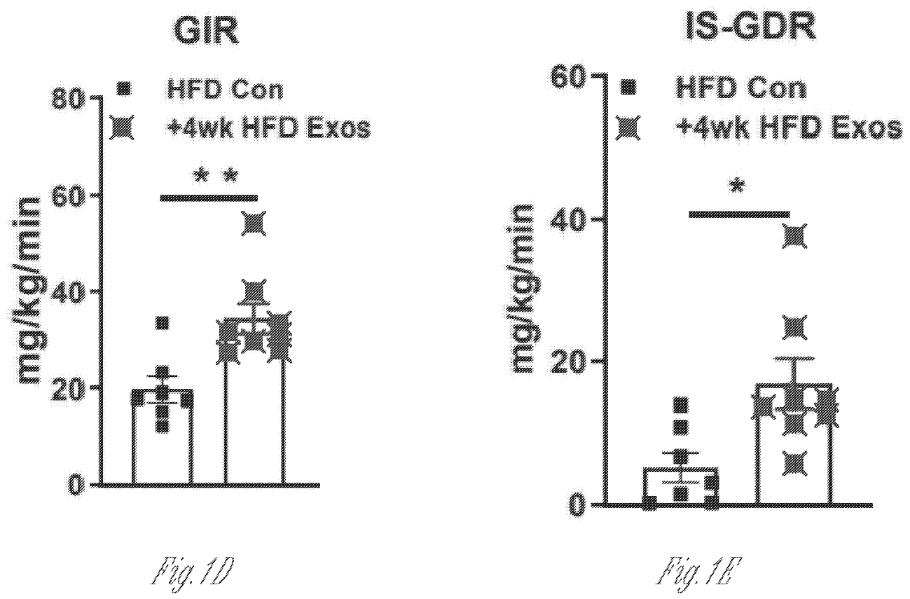

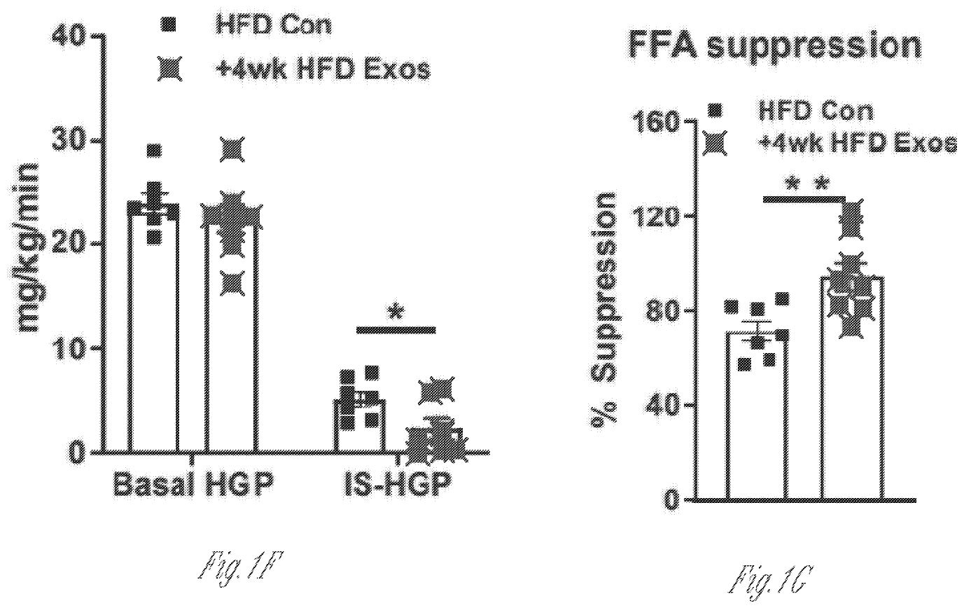

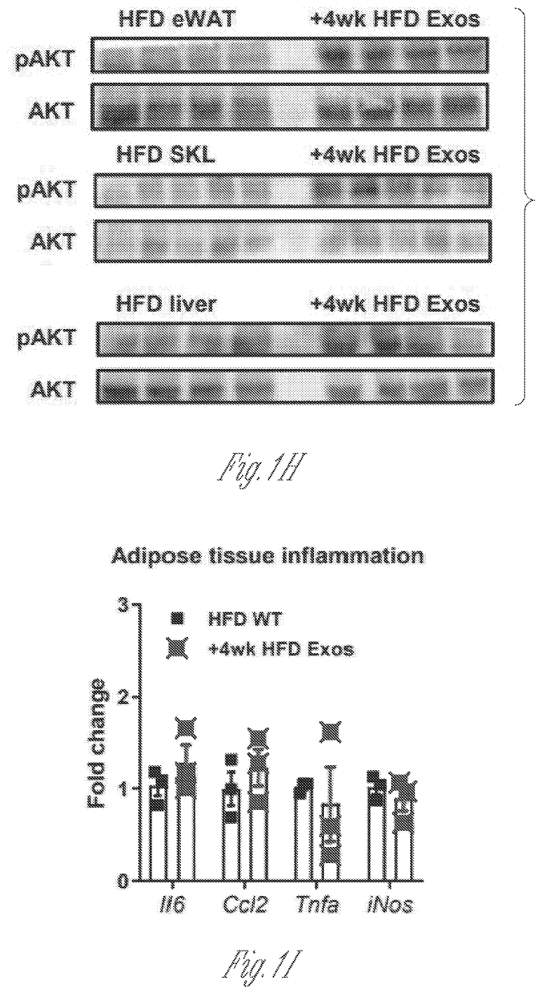

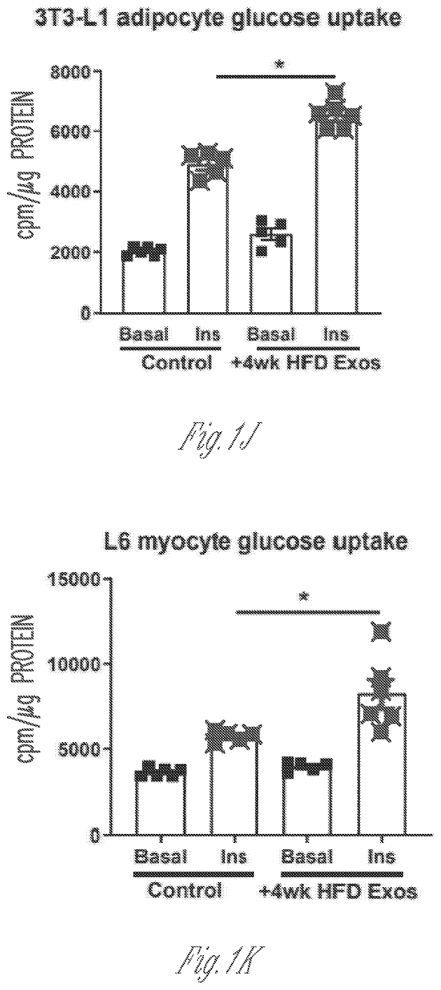

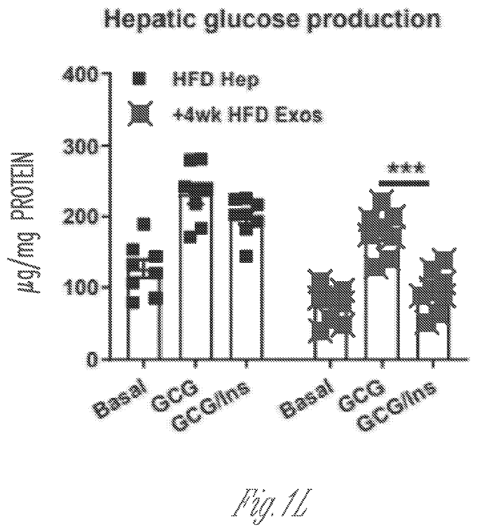

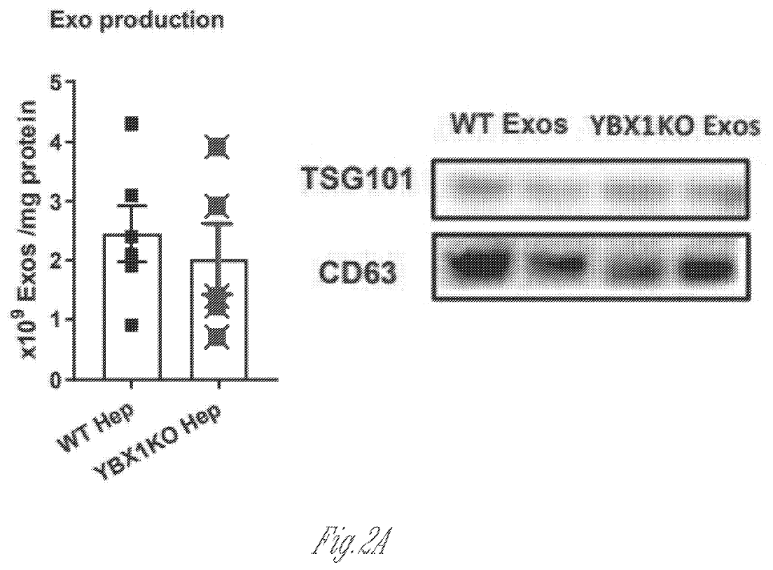

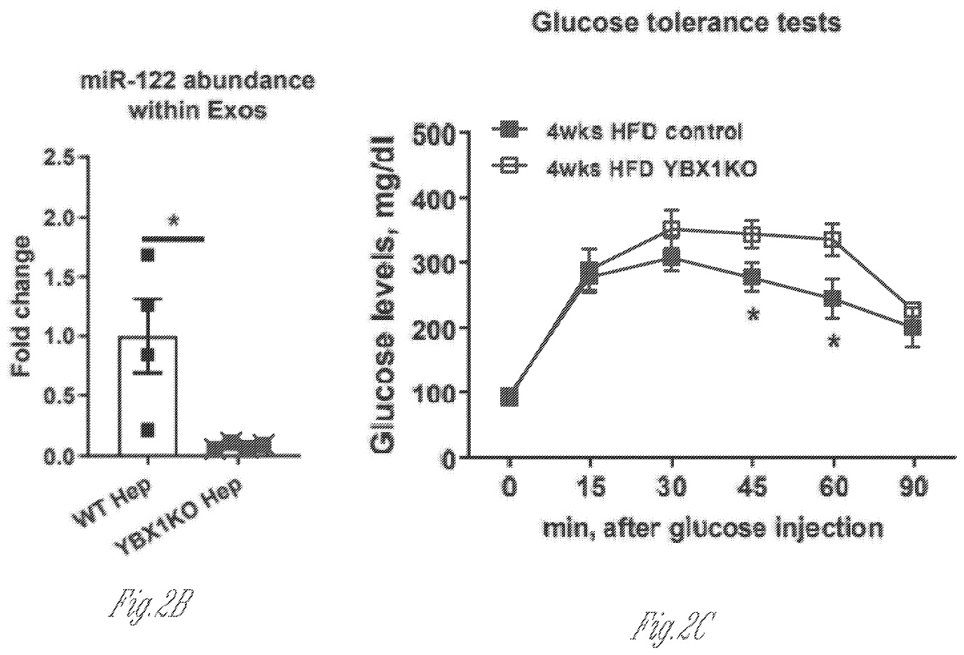

Exos-associated characteristics, such as protein markers and particle size, were similar across these hepatocyte Exos preparations. Hepatocytes from both 4 and 16 week HFD fed mice, secreted more Exos than hepatocytes from lean (NCD, normocaloric diet) mice (FIG. 1A). Sequencing of miRNA showed that the expression patterns differed within lean, early onset obesity 4-week HFD, and established obesity 16-week HFD hepatocyte-derived Exos. For example, hepatocytes of early onset obesity 4-week HFD mice secreted Exos that expressed more miR-3075-5p than Exos from lean or 16-week HFD mouse hepatocytes.

The 4 week HFD Exos were labeled with red fluorescent PKH26 dye and injected into 12 week HFD/obese mice. Hepatocyte Exos can be transferred and taken up by insulin target tissues, as evidenced by experiments demonstrating the appearance of red fluorescence in the liver, adipose tissue, and skeletal muscle of the 12 weeks HFD/obese recipient mice.

The metabolic effects of 4 week HFD hepatocyte Exos were tested. To do this, 4 week HFD Exos were intravenously injected into 12 week HFD/obese WT mice (5×10⁹ Exos/mouse, twice per week for 6 weeks). Additional groups of 12 week HFD/obese mice were treated with either empty liposomes (HFD Con) or hepatocyte Exos derived from chow-fed lean mice (Lean Exos). While all recipient groups displayed comparable body weights after Exos treatment, administration of 4 week HFD Exos led to improved glucose tolerance (FIG. 1B) and insulin sensitivity (FIG. 1C) compared to treatment with either empty liposomes (HFD control) or lean hepatocyte Exos (FIG. 1B-IC). The beneficial metabolic effects of 4 week HFD Exos treatment on in vivo insulin sensitivity was further assessed by performing hyperinsulinemic, euglycemic clamp studies. After treatment with 4 week HFD Exos, the recipient 16 wk HFD/obese mice exhibited higher glucose infusion rates, enhanced insulin-stimulated glucose disposal rate, and a greater degree of insulin-mediated suppression of hepatic glucose production and circulating free fatty acid levels (FIG. 1D-IG). These results show that 4 week HFD Exos can provide systemic insulin sensitivity in all major insulin target tissues. Consistent with this, the levels of insulin-stimulated AKT phosphorylation in skeletal muscle, liver, and adipose tissue were enhanced in the HFD/obese recipient mice treated with the 4 week HFD Exos (FIG. 1H). Interestingly, 4 week HFD Exo treatment had minimal effects on adipose tissue inflammation, as exemplified by similar levels of M1 and M2-like ATMs. Adipose tissue proinflammatory cytokine expression in the chronically obese recipient HFD mice treated with 4 week HFD Exos were the same compared to empty liposome-treated controls (FIG. 1I).

The direct effects of 4 wk HFD Exos on in vitro insulin signaling in adipocytes, myocytes, and hepatocytes were assessed. Consistent with the improvement induced by 4 week HFD Exos on in vivo insulin sensitivity, 3T3-L1 adipocytes and L6 myocytes exhibited increased insulin-stimulated glucose uptake after treatment with the 4 week HFD Exos (1×10⁸ Exos per 0.5×10⁶ cells; FIG. 1J-1K). In addition, primary hepatocytes isolated from 12 week HFD/obese mice that were treated with Exos from 4 week HFD mouse hepatocytes led to enhanced insulin-mediated suppression of glucagon-stimulated glucose production (GCG-Ins), compared to the insulin-resistant obese hepatocytes treated with empty liposomes (GCG, FIG. 1L). In addition, 4 week HFD Exos treatment led to increased levels of insulin-stimulated AKT phosphorylation in adipocytes, myocytes, and hepatocytes.

Taken together, these results indicate that 4 week HFD Exos directly mitigate obesity-associated insulin resistance.

### Example 3: Inhibition of Hepatocyte Exos Secretion and miRNA Loading

To deplete exosomal miRNAs, hepatocyte-specific Y-box-binding protein 1 (YBX1) knockout (KO) mice were generated. YBX1 is a component of the machinery that loads miRNAs into Exos (Shurtleff et al., 2016; Shurtleff et al., 2017). To knockout YBX1 in hepatocytes, hepatocyte-specific Cas9 transgenic mice were treated with a lentivirus (1×10⁸ particles/mouse) carrying a YBX1 guide RNA (gRNA-YBX1) designed to delete the translational start site (Ran et al., 2015; Platt et al., 2014). YBX1 was not detected in hepatocytes from the YBX1 knockout mice by immunoblotting.

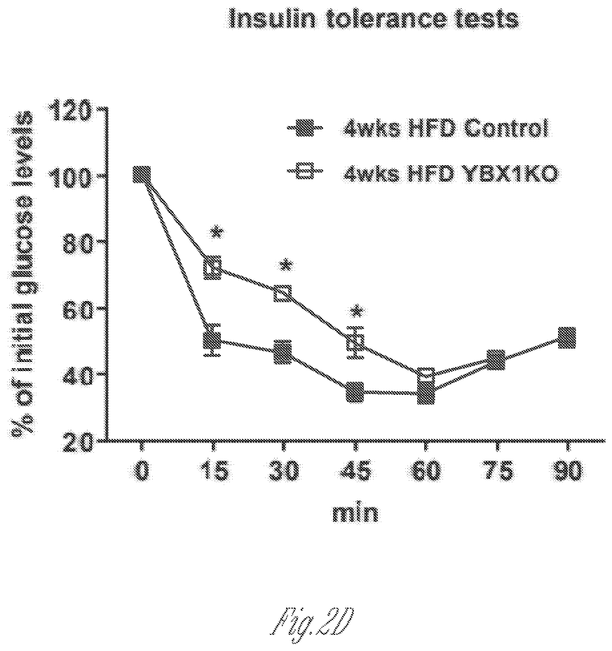

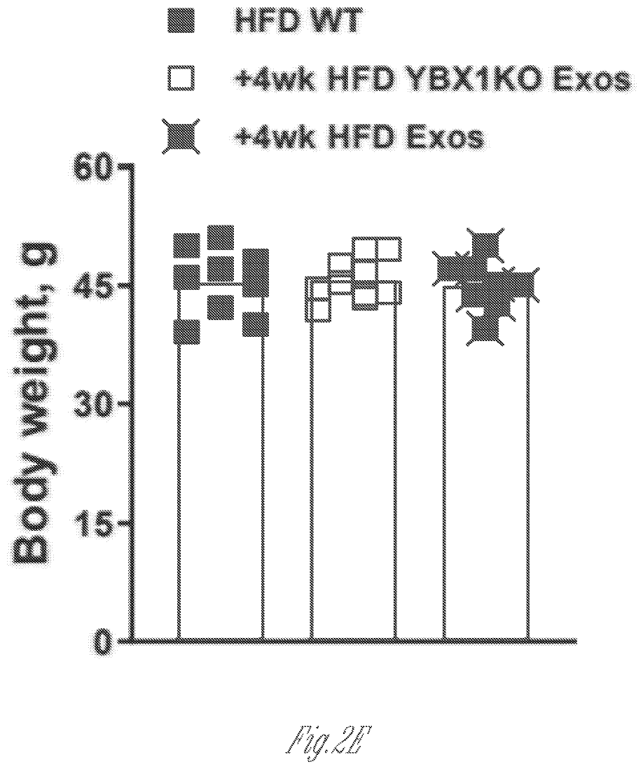

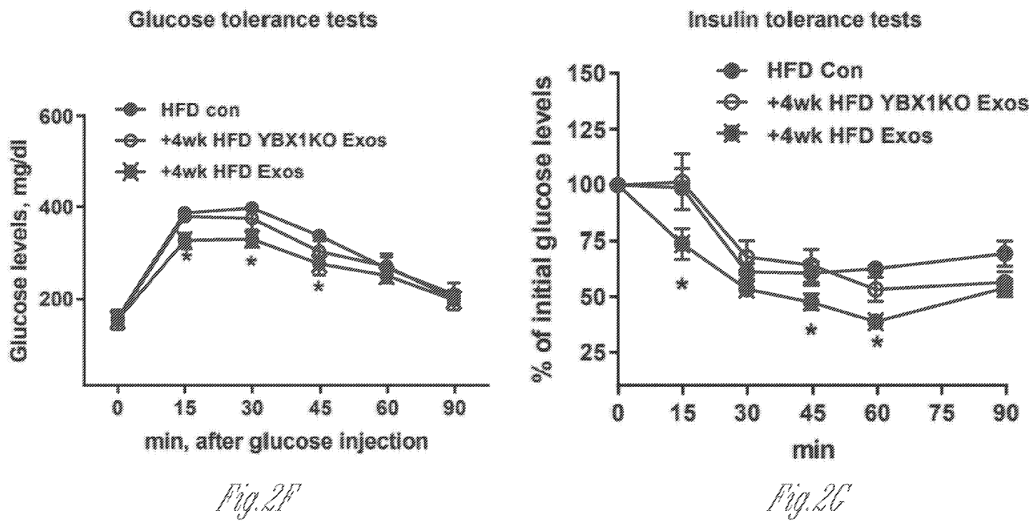

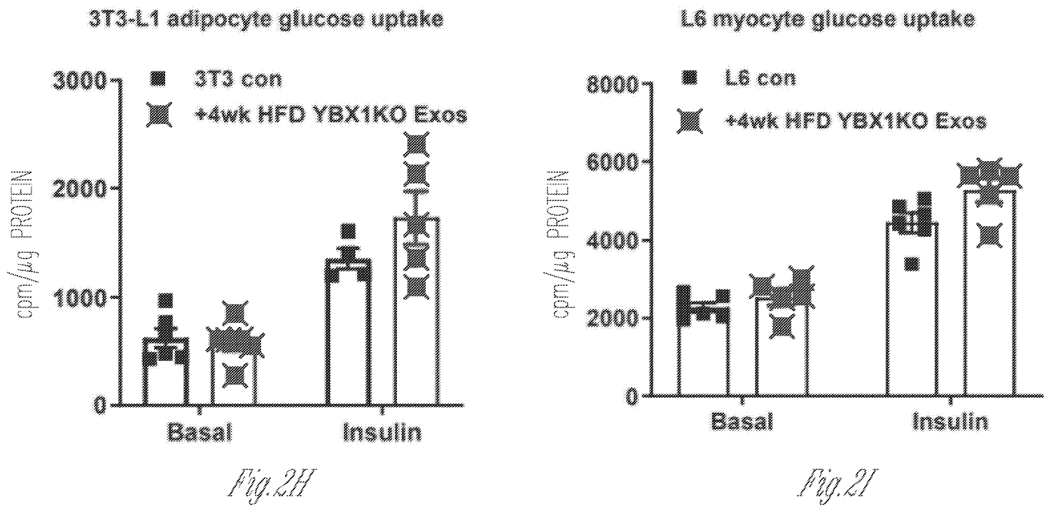

Deletion of YBX1 did not affect hepatocyte Exos production or the expression of Exos-associated protein markers such as TSG101 (FIG. 2A) but led to a marked decrease in the miRNA content within the hepatocyte Exos, as shown by the >95% reduction in miR-122 (a highly expressed miRNA in hepatocytes) abundance (FIG. 2B). As seen in FIG. 2C-2D, hepatocyte YBX1 deletion exacerbated glucose and insulin tolerance in 4 week HFD KO mice. However, no change in body weight was observed (FIG. 2E). YBX1-KO mediated miRNA depletion therefore abolished the beneficial effects of 4 week HFD Exos on glucose tolerance and in vivo insulin sensitivity and did not affect body weight. This is further illustrated by similar glucose and insulin tolerance tests between 18 weeks HFD WT recipient mice treated either with 4 week HFD YBX1-KO hepatocyte Exos or empty liposomes compared to the improvement in 4 week HFD Exos treated obese mice (FIG. 2F-2G). Four week HFD YBX1-KO Exos treatment also had no significant effects on glucose uptake in 3T3-L1 adipocytes or L6 myocytes (FIG. 2H-2I).

Previous studies have indicated that Rab27, a small GTPase, is important for the secretion of small vesicles such as exosomes (Ostrowski et al., 2010; Song et al., 2019; Bobrie et al., 2012). To evaluate the importance of Rab27 on the release of Exos and extracellular miRNAs from hepatocytes, a hepatocyte-specific Rab27 knockout (Rab27-KO) mouse model was generated. This was done by intravenous injection of a lentivirus harboring the guide RNAs for start site deletion of both Rab27a and Rab27b (gRNA-Rab27a and gRNA-Rab27b, respectively) into hepatocyte-specific Cas9 transgenic mice. Control Cas9 transgenic mice were injected with a lentivirus carrying control vectors without gRNA. Western analysis of hepatocytes from Rab27-KO mice indicated that little or no Rab27 was detectable.

Nanoparticle analysis confirmed a significant reduction in hepatocyte Exos secretion after Rab27 depletion (FIG. 3A). More importantly, deletion of Rab27 in hepatocytes significantly reduced the miRNA content of the remaining EVs, as evidenced by an about 90% lower level of miR-122 per 1×10⁹ EVs (FIG. 3B). Most likely, this is because many of the remaining “Exos” are other forms of EVs which do not carry miRNAs. EVs derived from 4 week HFD Rab27KO hepatocytes did not affect in vitro hepatic glucose production (FIG. 3C), in contrast to treatment with 4 week HFD Exos from WT mice. After 4 week HFD feeding, hepatocyte-specific Rab27KO mice had comparable body weight to control Cas9+ mice injected with lentivirus carrying control vectors. However, hepatocyte Rab27 depletion resulted in glucose intolerance and insulin resistance, compared to the WT control mice (FIG. 3D-3E). Lower insulin-stimulated AKT phosphorylation was observed in liver, skeletal muscle, and adipose tissue in hepatocyte-specific Rab27KO mice compared to WT control mice (FIG. 3F). In addition, hepatocytes isolated from 4 week HFD hepatocyte-specific Rab27KO mice displayed impaired insulin suppression of glucagon-induced glucose output, whereas 4 week HFD WT hepatocytes showed normal insulin sensitivity (FIG. 3G). These data indicate that endogenous miRNAs within 4 week HFD hepatocytes provide signals needed to mitigate cellular insulin resistance. Overall, the results indicate that expression of insulin sensitizing miRNAs in 4 week HFD 210 hepatocyte Exos may be a compensatory response in an attempt to mitigate HFD/obesity-induced insulin resistance.

### Example 4: miR-3075 in 4 Week HFD Exos Produces Insulin Sensitivity

The mechanisms by which miRNAs within 4 week HFD Exos promote insulin sensitivity were explored in this Example.

MiRNA sequencing analysis had revealed that 4 week HFD feeding induced a set of miRNAs uniquely enriched in hepatocyte Exos. This increased expression was greatest and most consistent for miR-3075. In addition, it was validated that miR-3075 was highly expressed in key metabolic tissues after treatment with 4 week HFD Exos, demonstrating transfer and uptake from these Exos to adipose tissue, skeletal muscle, and liver (FIG. 4A-4C). In contrast, treatment with 4 week HFD YBX1-KO Exos did not affect miR-3075 abundance in obese recipient mice (FIG. 4A-4C). Interestingly, at 4 weeks on a HFD, primary hepatocytes maintained a comparable level of insulin sensitivity as primary hepatocytes from chow-fed lean mice (FIG. 4D-4F), demonstrating that they do not exhibit cell-autonomous insulin resistance. These cells express significant endogenous miR-3075 (FIG. 4H).

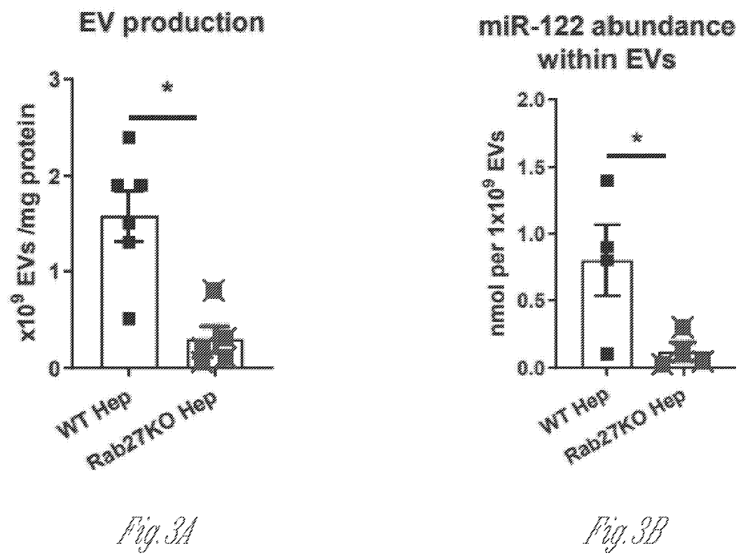

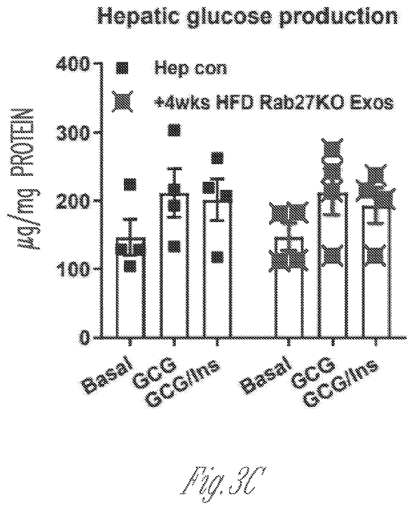

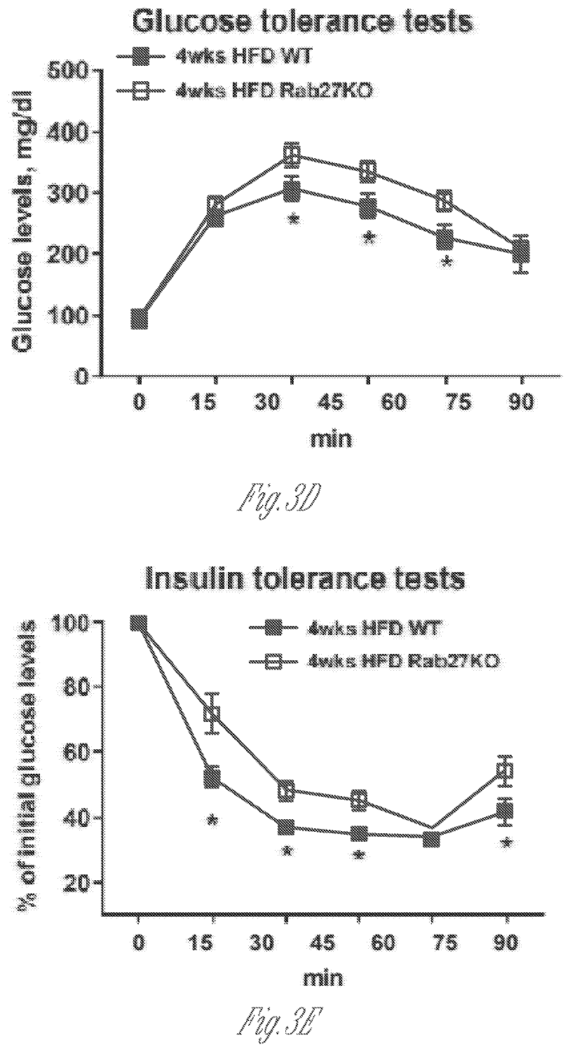

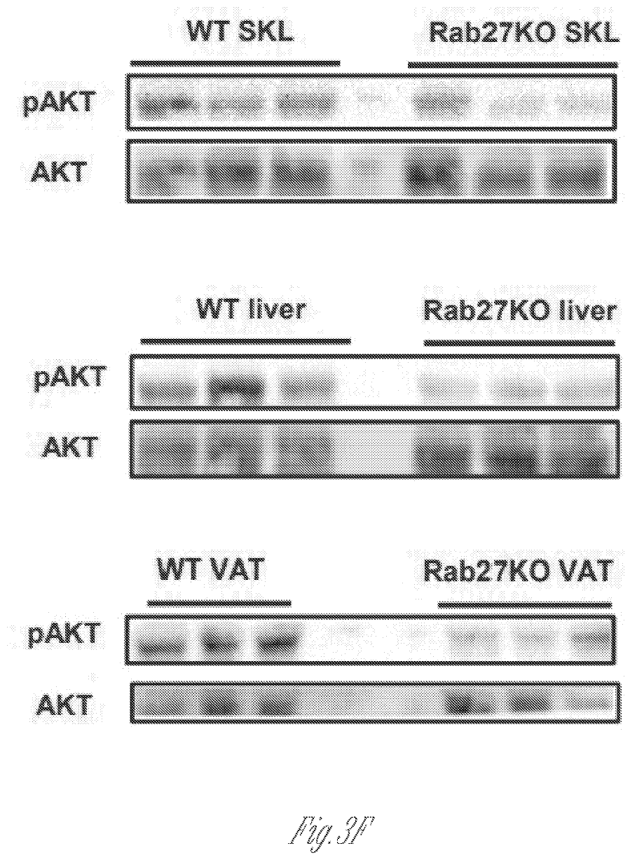

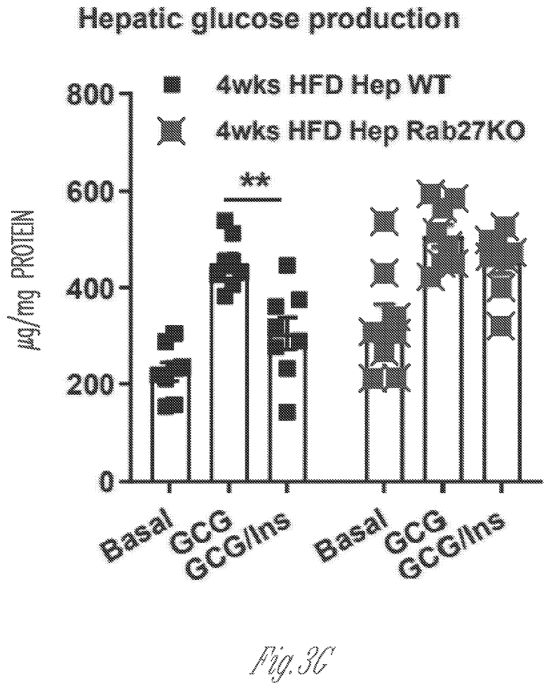

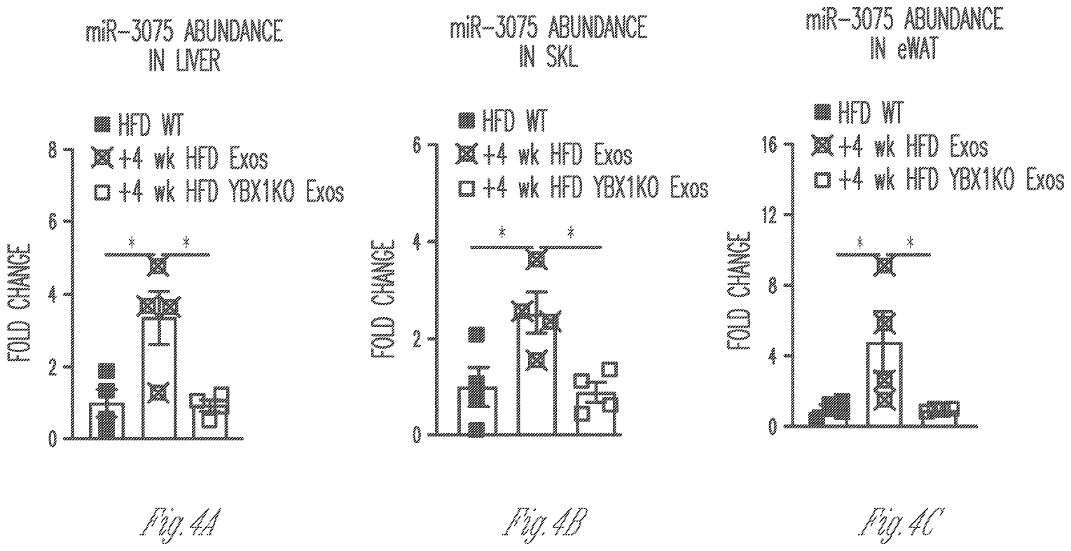

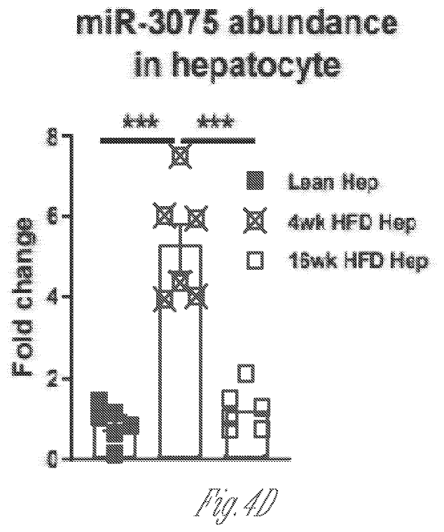

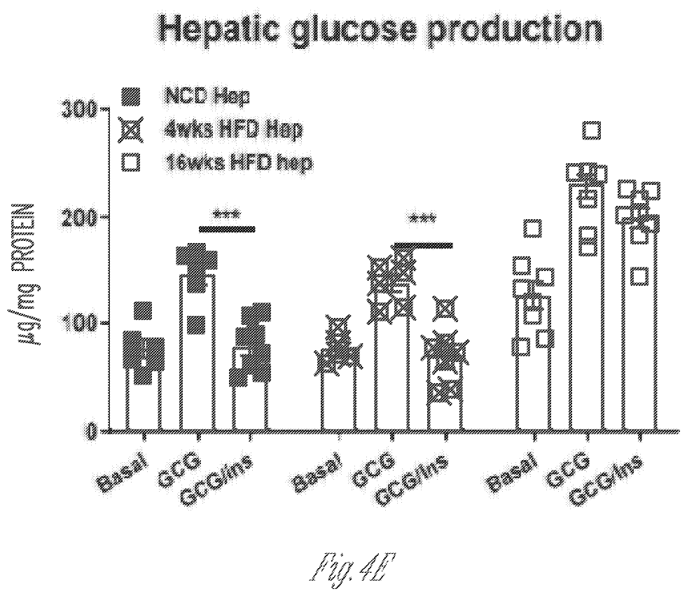

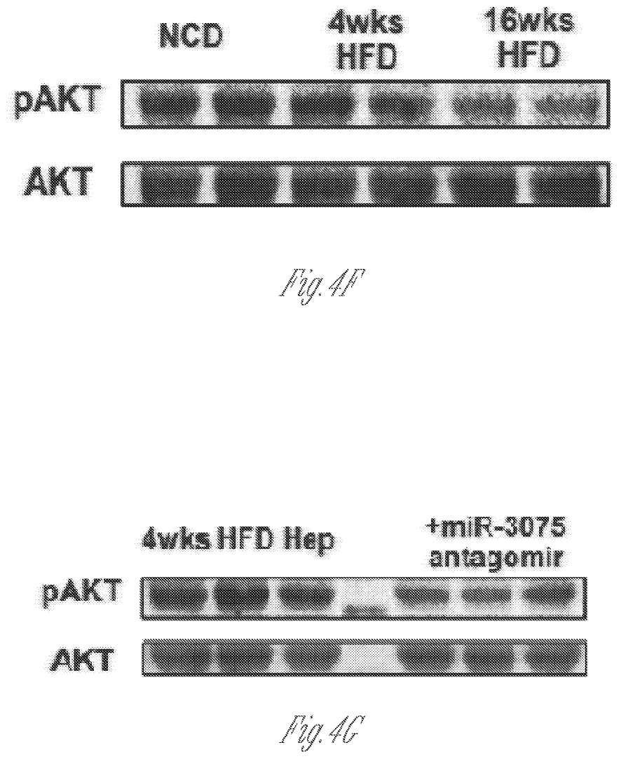

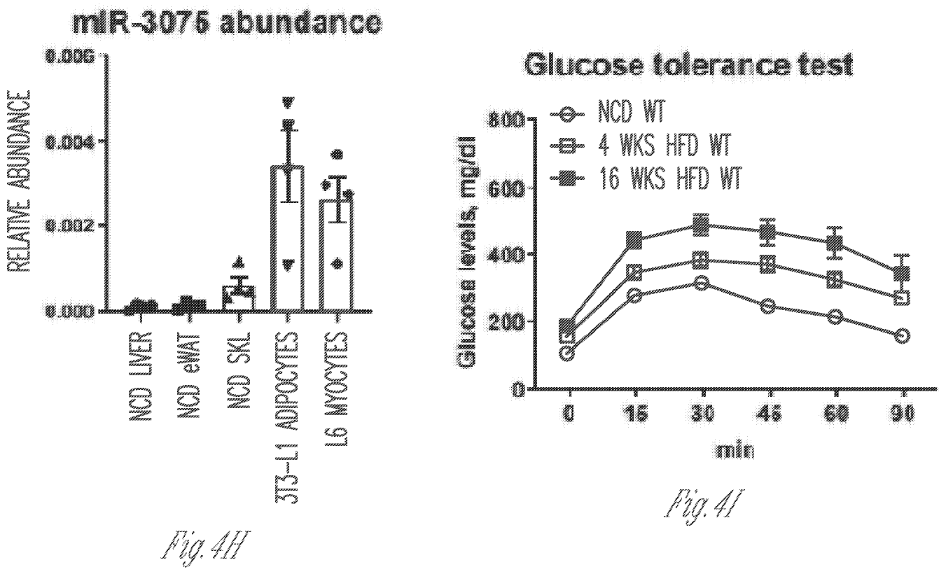

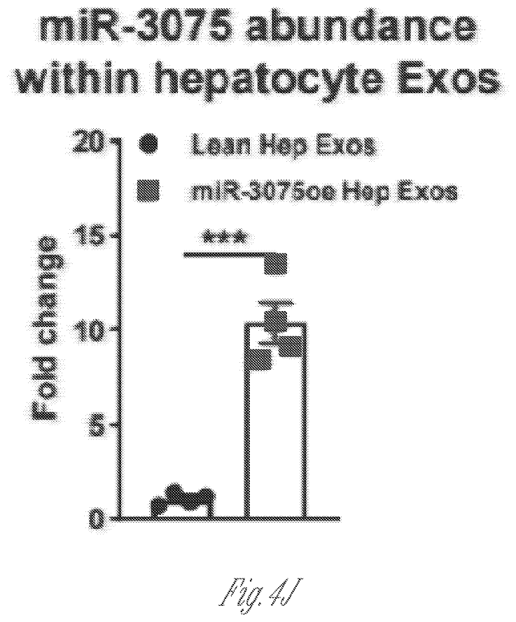

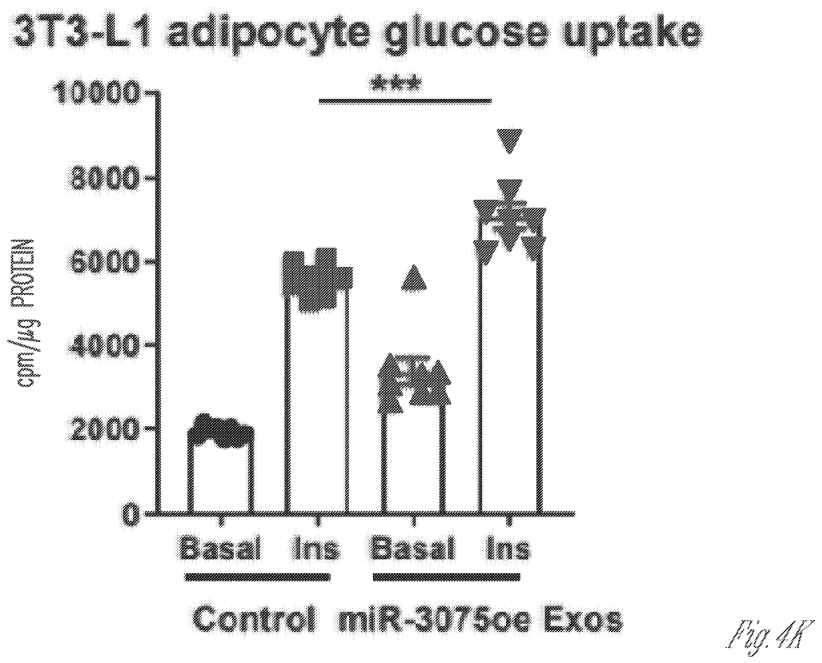

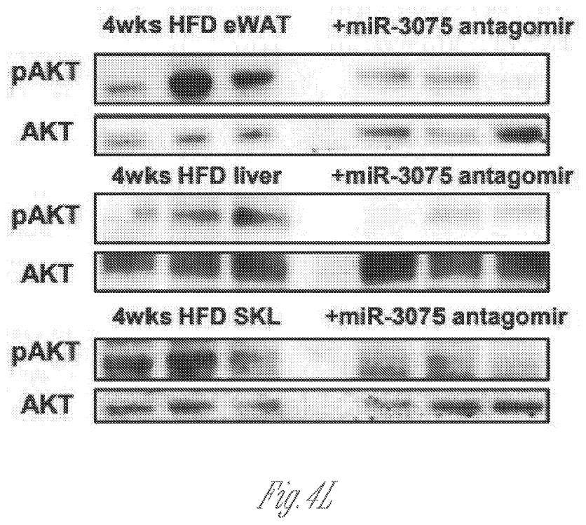

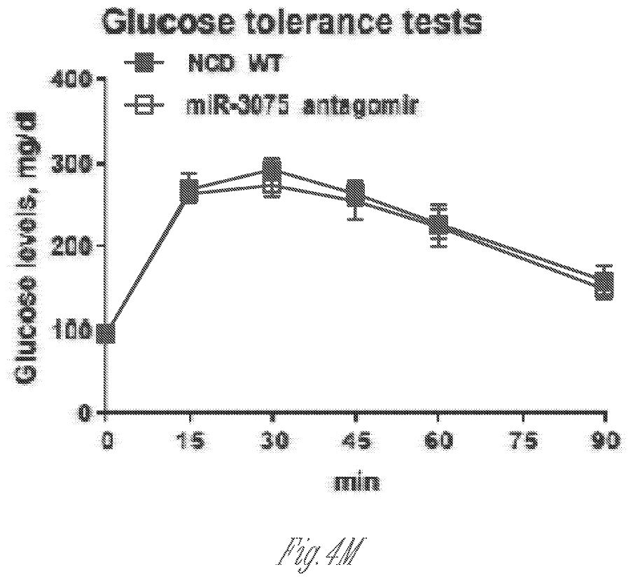

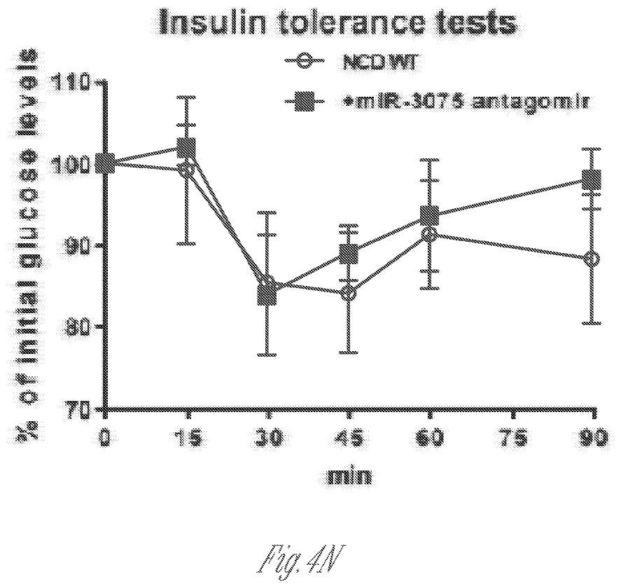

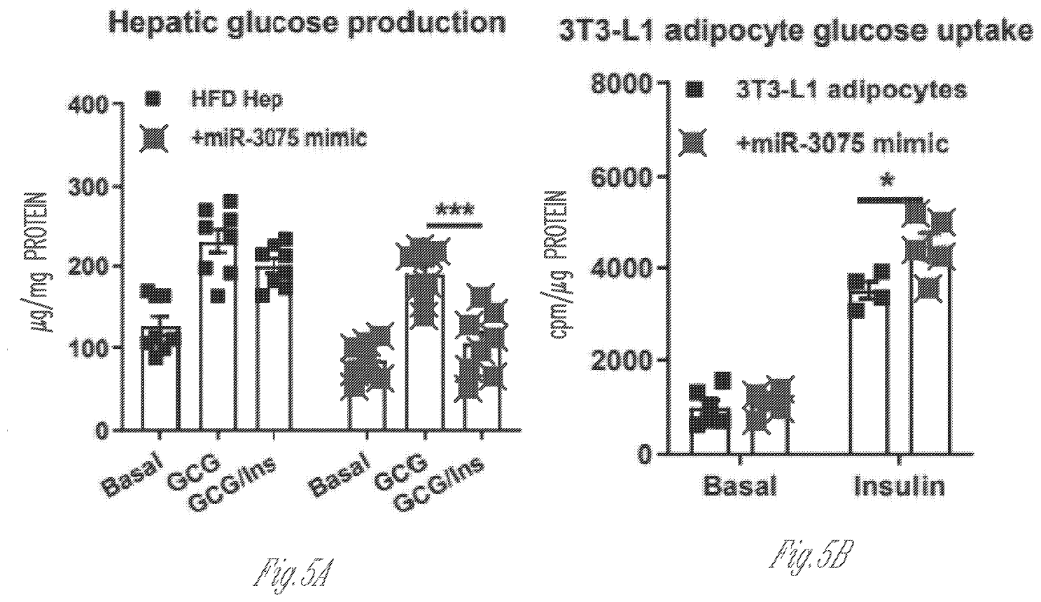

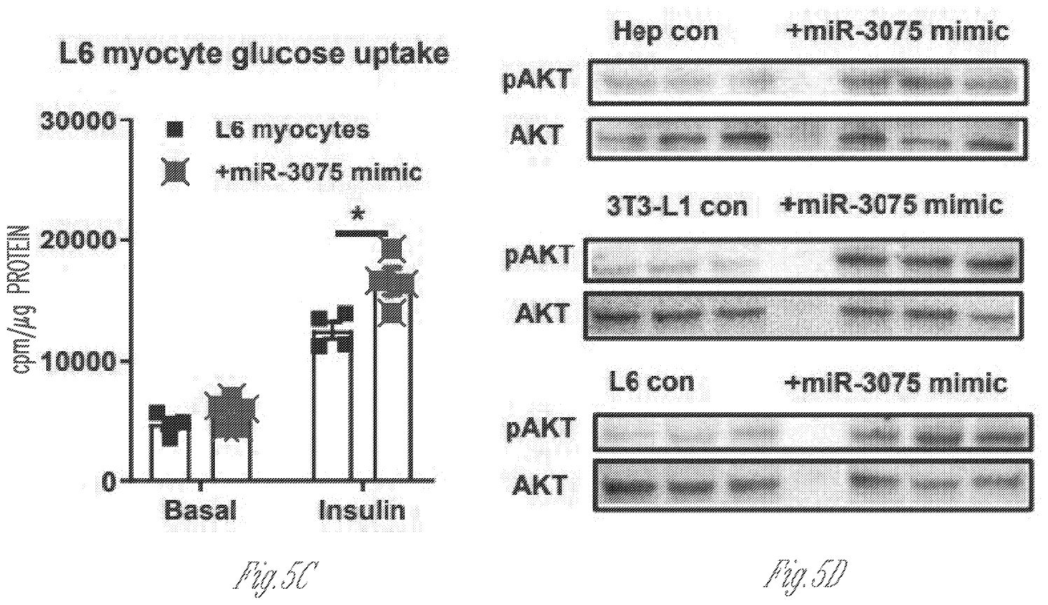

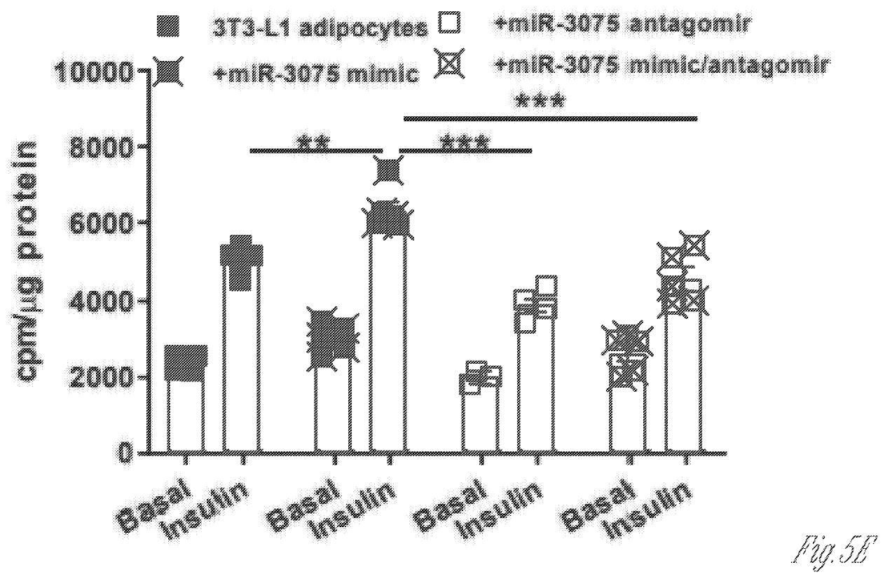

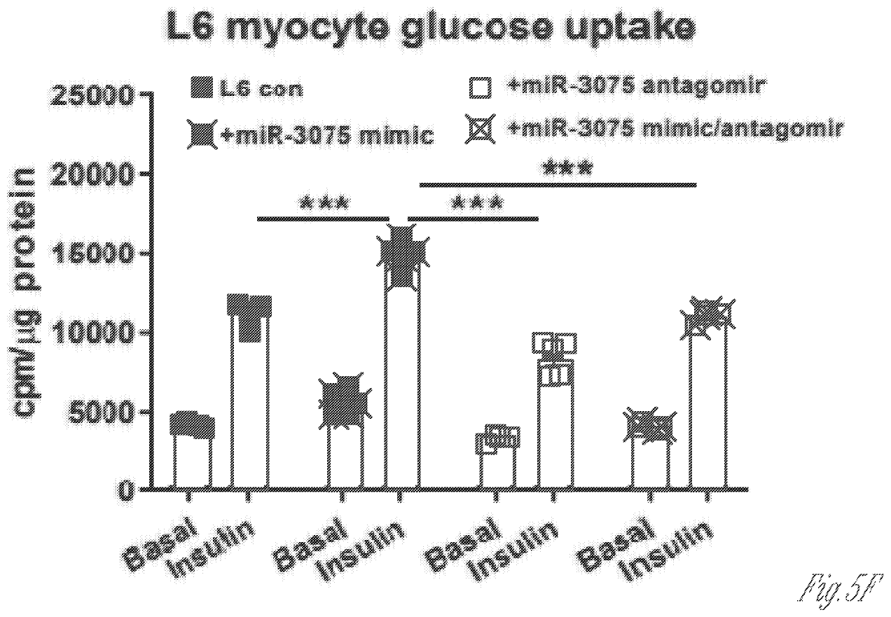

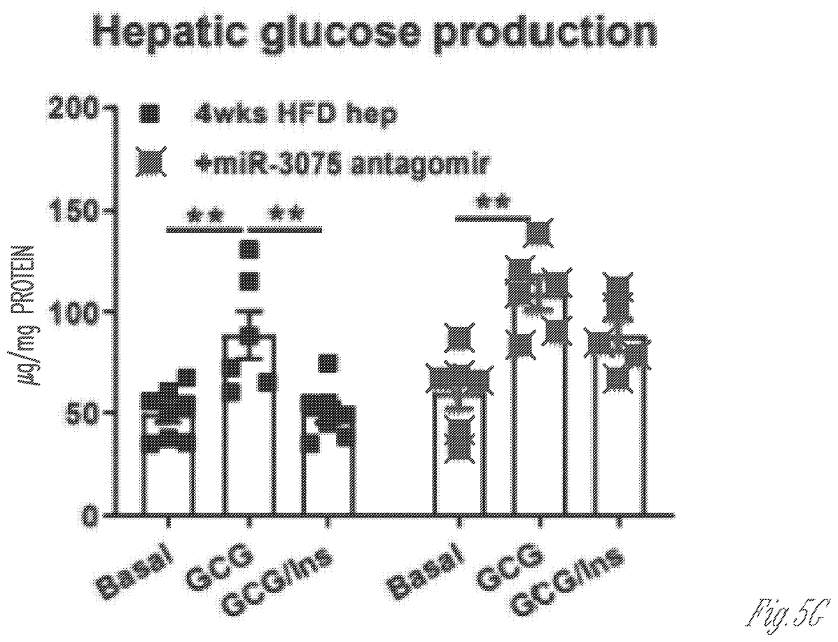

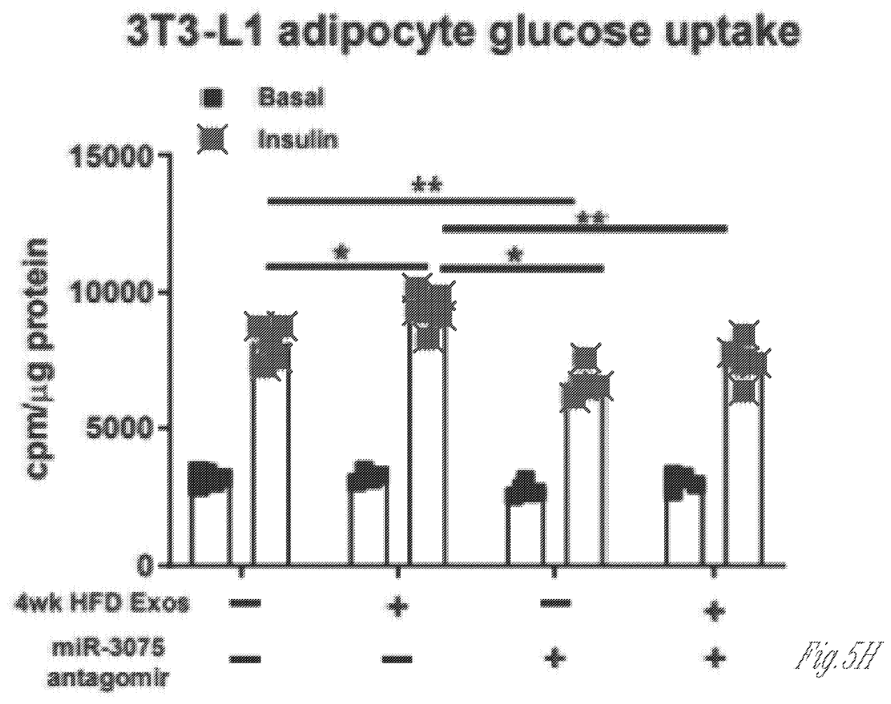

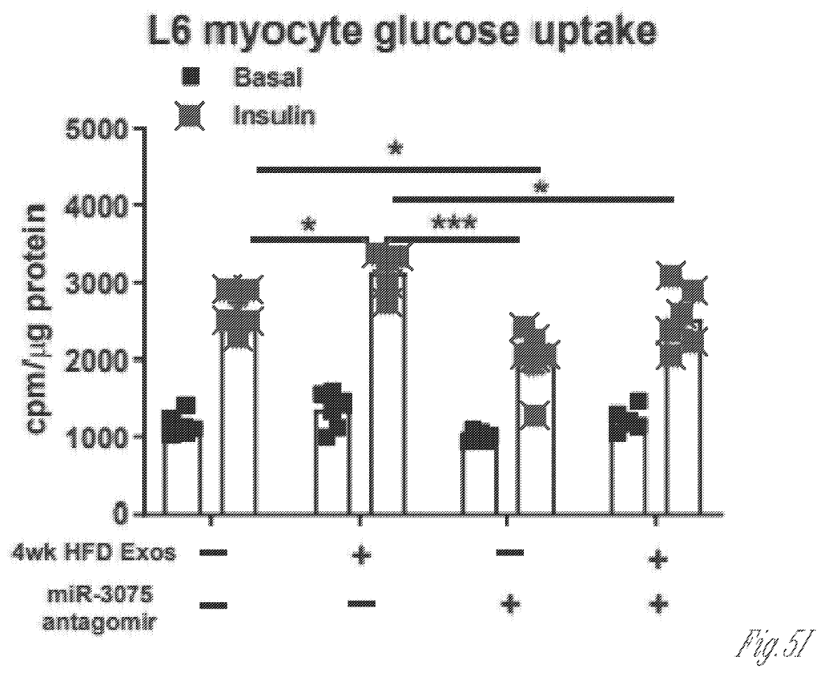

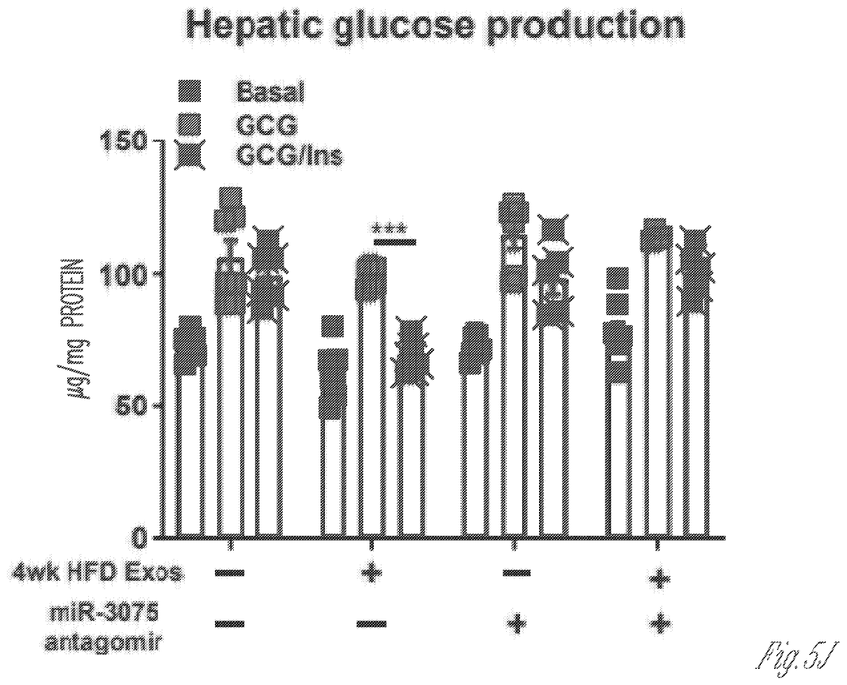

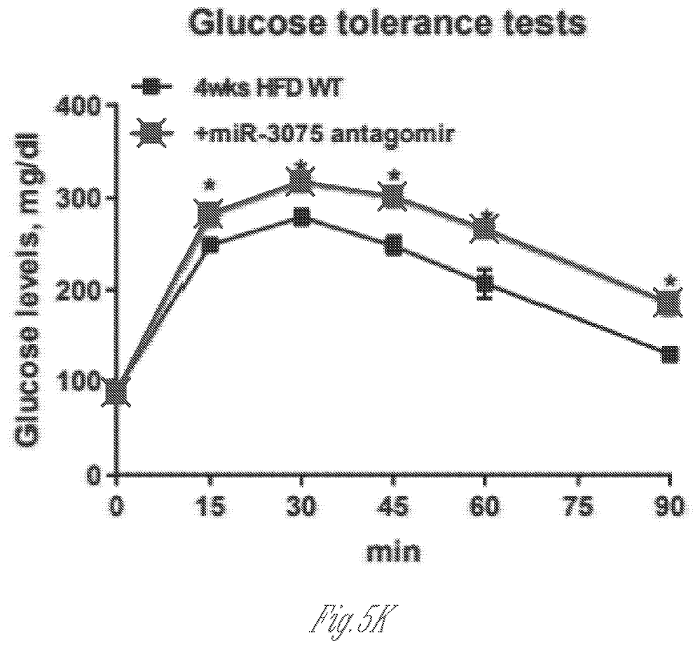

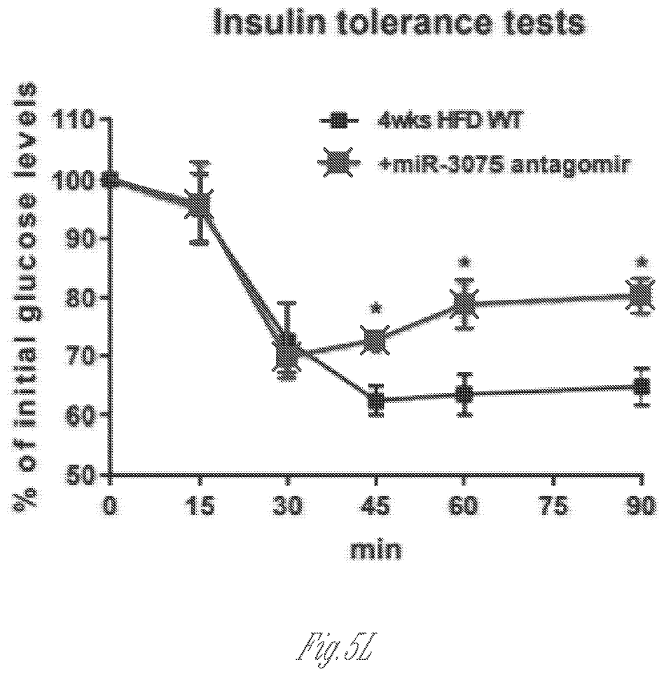

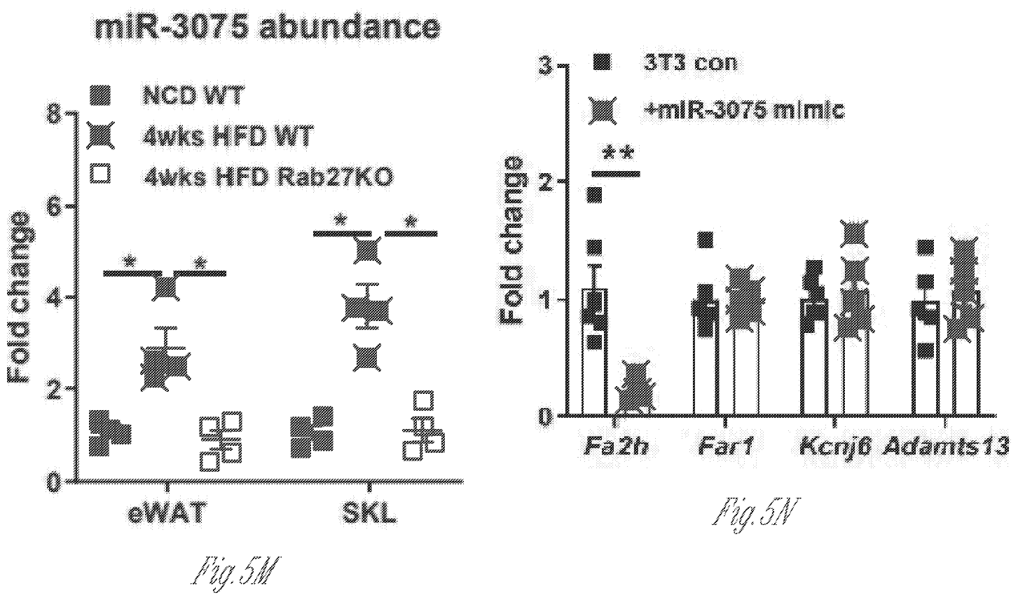

These results led the inventors to hypothesize that miR-3075 plays a role in attenuating the full obesity-induced insulin resistance in early onset obesity. To assess this, a miR-3075 mimetic packaged into artificial liposomes was incubated with primary hepatocytes from 12 week HFD/obese mice. As shown in FIG. 5A, insulin-resistant primary hepatocytes obtained from 12 weeks HFD/obese mice exhibited restored normal insulin sensitivity when treated with the miR-3075 mimetic packaged into artificial liposomes. In addition, treatment of 3T3-L1 adipocytes and L6 myocytes with the miR-3075 mimic-containing liposomes led to enhanced adipocyte and myocyte insulin-stimulated glucose uptake (FIG. 5B-5C), as well as greater levels of insulin-stimulated phosphorylated AKT (FIG. 5D). Furthermore, co-treatment with a miR-3075 antagomir (siRNA-FA2H) plus the miR-3075 mimic blocked this improvement in insulin-stimulated glucose uptake in 3T3-L1 adipocytes and L6 myocytes (FIG. 5E-5F). Importantly, the miR-3075 antagomir alone led to impaired insulin signaling in these three cell types (FIG. 5E-5F; FIG. 4G), showing that endogenous levels of miR-3075 participate in maintaining a high state of insulin signaling in these normal cells (FIG. 5H-5J).

Chronically obese 16 week HFD mice displayed markedly impaired glucose tolerance compared to lean mice, while glucose tolerance in 4 week HFD mice was intermediate (FIG. 4I). Hepatocytes from lean mice were also transfected with the miR-3075 mimic to further enrich this miRNA in the hepatocyte Exos derived from these lean mice (miR-3075oe Exos) (FIG. 4J). As expected in view of the other results described herein, treatment with these miR-3075 enriched Exos led to increased insulin-stimulated glucose uptake in 3T3-L1 adipocytes from chronically obese 16 week HFD (FIG. 4K).

To further evaluate the importance of miR-3075 on the effect of 4 week HFD Exos, obese hepatocytes and 3T3-L1 adipocytes were treated with a combination of 4 week HFD Exos and the miR-3075 antagomir. As shown in FIG. 5H-5J, treatment with 4 week HFD Exos enhanced cellular insulin action, whereas the miR-3075 antagomir blocked the effects of 4 week HFD Exos.

The effects of a miR-3075 antagomir were also assessed in vivo during early stage obesity. Wild type mice were intravenously injected with liposomes encapsulating miR-3075 antagomir (2× per week, 10 nmole/mouse; starting at 1 week of HFD). After 4 weeks, miR-3075 antagomir treatment led to impaired glucose tolerance and insulin resistance compared to control mice treated with empty liposomes (FIG. 5K-5L). Lean WT mice who have normal glucose tolerance displayed no changes in metabolic responses in response to miR-3075 antagomir treatment (FIG. 4M-4N). Indeed, miR-3075 was expressed at low levels in the key metabolic tissues of lean mice (FIG. 4H), consistent with the absence of antagomir effects. Together, these results show that miR-3075 is the main contributor to the insulin-sensitizing effects of 4 week HFD Exos.

Taken together, these results indicate that in early stage obesity hepatocytes produce Exos enriched in insulin sensitizing miR-3075. These 4 week HFD hepatocyte Exos are secreted into the circulation where they engage distal insulin target cells mitigating insulin resistance, likely as a compensatory response to the initial HFD.

Because miR-3075 levels are quite low in tissues from chow-fed or chronically obese mice, the inventors hypothesize that the adipose tissue and skeletal muscle levels of miR-3075 at 4 week of a HFD are not endogenously produced but are derived from the circulating 4 week hepatocyte Exos. To test this hypothesis, miR-3075 abundance was evaluated in eWAT and skeletal muscle in 4 week HFD mice. As shown in FIG. 4D and FIG. 5M, miR-3075 abundance is elevated in eWAT from mice fed a HFD for 4 weeks compared to the results in lean chow-fed mice or chronically obese mice. In contrast, there were very low levels of miR-3075 in adipose tissue or skeletal muscle in 4 week HFD hepatocyte-specific Rab27-KO mice (FIG. 5M). These data indicate that hepatocytes can be the source of miR-3075 in adipose tissue and skeletal muscle at 4 week HFD.

### Example 5: Fatty Acid 2-Hydroxylase is a miR-3075 Target Gene that can Regulate Insulin Signaling

miRNAs exert their biological effects by causing translational arrest and/or inducing degradation of their target miRNAs through base pairing between the miRNA seed sequence and the target mRNA recognition site. A target gene prediction algorithm, Target Scan Mouse 7.2, was used to identify theoretical target genes of miR-3075 (Agarwal et al., 2015). A group of mRNAs were identified that harbor this target site sequence in their 3′ untranslated region (UTR; FIG. 5N) that included 2-Hydroxylase (FA2H), Fatty Acyl-CoA Reductase 1 (FAR1), Potassium Inwardly Rectifying Channel Subfamily J Member 6 (KCNDJ6), and ADAM Metallopeptidase With Thrombospondin Type 1 Motif 13 (ADAMTS13).

After validating, the abundance of the gene encoding fatty acid 2-Hydroxylase (FA2H) was evaluated in various cell types when miR-3075 mimic-containing liposomes were present. As shown in FIG. 5N, FA2H protein levels were markedly downregulated after treatment of adipocytes, hepatocytes and L6 myocytes with the miR-3075 mimic-containing liposomes.

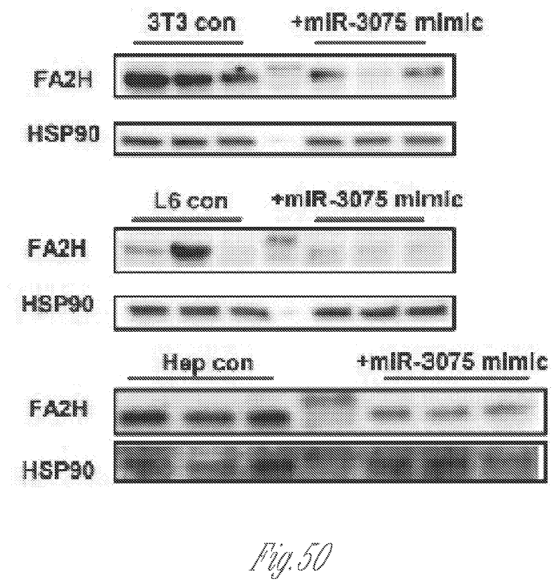

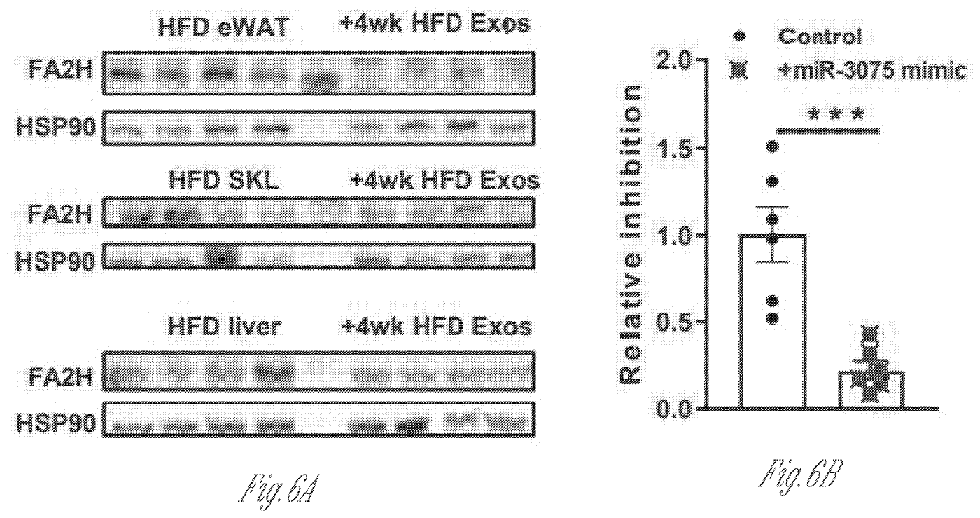

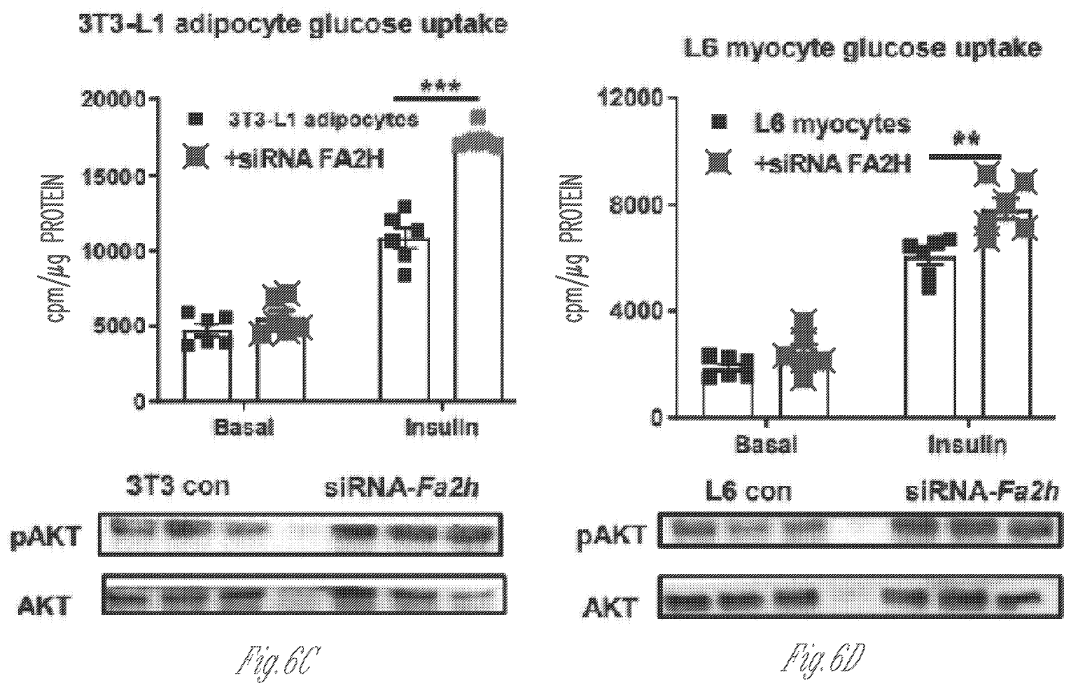

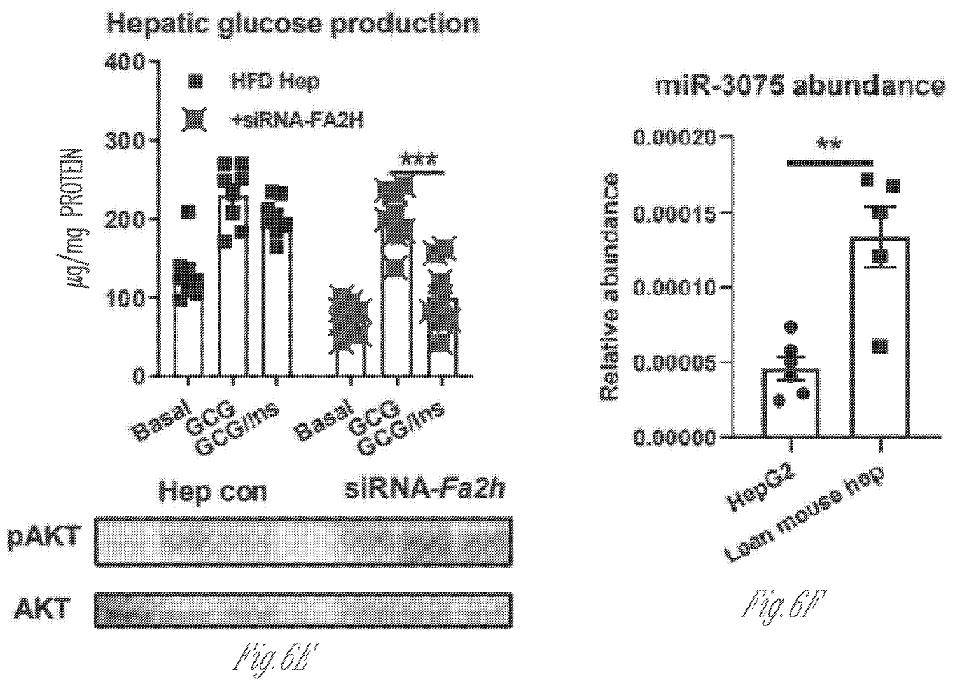

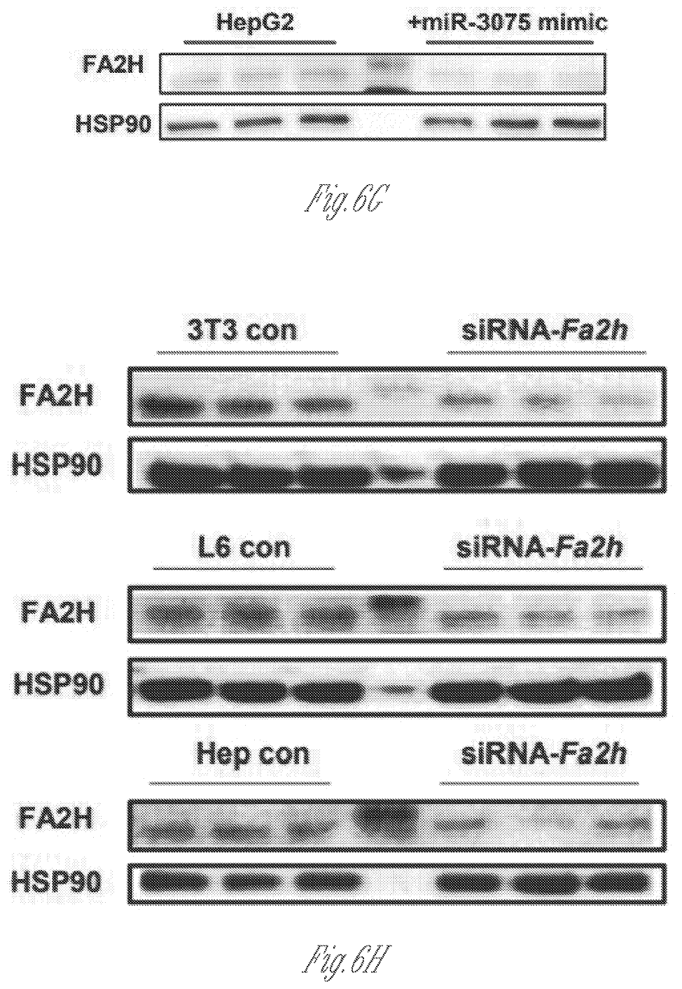

In view of such in vitro data, FA2H expression was evaluated in vivo within eWAT, skeletal muscle, and liver in 16 weeks HFD/obese mice after treatment with 4 week HFD Exos. As seen in FIG. 6A, FA2H protein abundance was markedly reduced in these metabolic tissues following the treatment period.

To further confirm that Fa2h is a genuine target mRNA of miR-3075, luciferase reporter assays were performed with a vector carrying the Fa2h 3′ UTR. After 24 hours co-transfection into HEK293 cells of this reporter construct and the miR-3075 mimic, luciferase activity was decreased by about 90% compared to control cells transfected with only the Fa2h 3′ UTR vector (FIG. 6B). It was observed that miR-3075 was expressed in human HepG2 cells (FIG. 6F). More importantly, overexpression of miR-3075 also led to less FA2H abundance in human HepG2 cells (FIG. 6G). These results further confirm that Fa2h mRNA is a target of miR-3075, and that miR-3075/miR-3075 mimics have the same effects on human cells as mouse cells.

To assess the importance of miR-3075-mediated Fa2h suppression on insulin signaling, a siRNA directed against Fa2h was utilized. As demonstrated in FIG. 6C-6D and FIG. 5N, knockdown (KD) of Fa2h led to increased insulin-stimulated glucose uptake in 3T3-L1 adipocytes and L6 myocytes, and this was accompanied by increased insulin-stimulated AKT phosphorylation. Furthermore, knockdown of Fa2h in insulin-resistant primary hepatocytes obtained from 12 week HFD/obese mice (GCG/Ins) led to normalization of insulin sensitivity (suppression of glucagon stimulated glucose production) compared to control cells transfected with a scrambled siRNA (FIG. 6E, 6H). Overall, these results support a conclusion that the miR-3075-Fa2h axis contributes to mediation of insulin sensitivity.

### Example 6: Prolonged HFD Feeding Induces Hepatocytes to Secrete Pathogenic Exos that Cause Tissue Inflammation and Insulin Resistance

RNA sequencing analysis showed that 16 weeks of a high fat diet (HFD) induced a group of miRNAs specifically enriched in hepatocyte Exos, compared to either a normocaloric diet (NCD) or 4 week of a HFD. The effect of these 16 week HFD hepatocyte-derived exosomal miRNAs were assessed on obesity-associated metabolic dysfunction by studies of hepatocyte-specific knockout of Rab27 or knockout of YBX1 in mice.

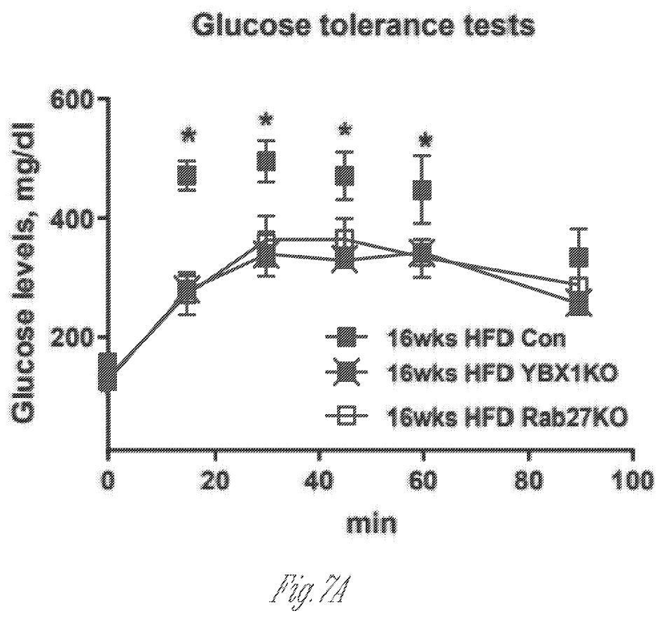

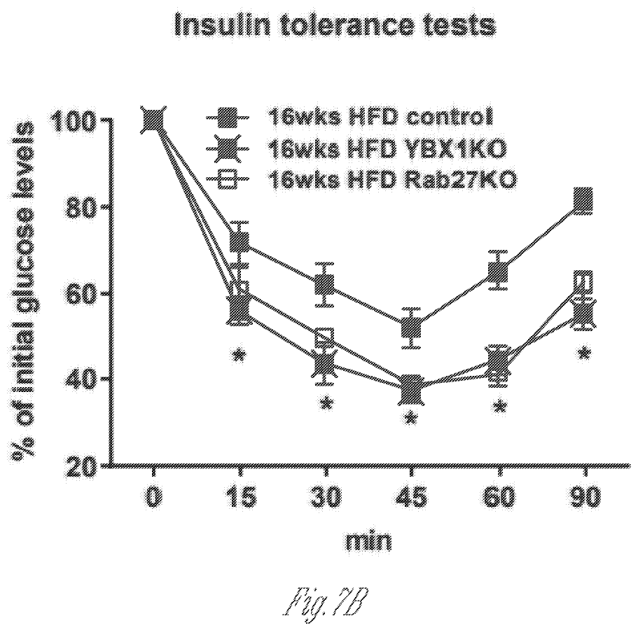

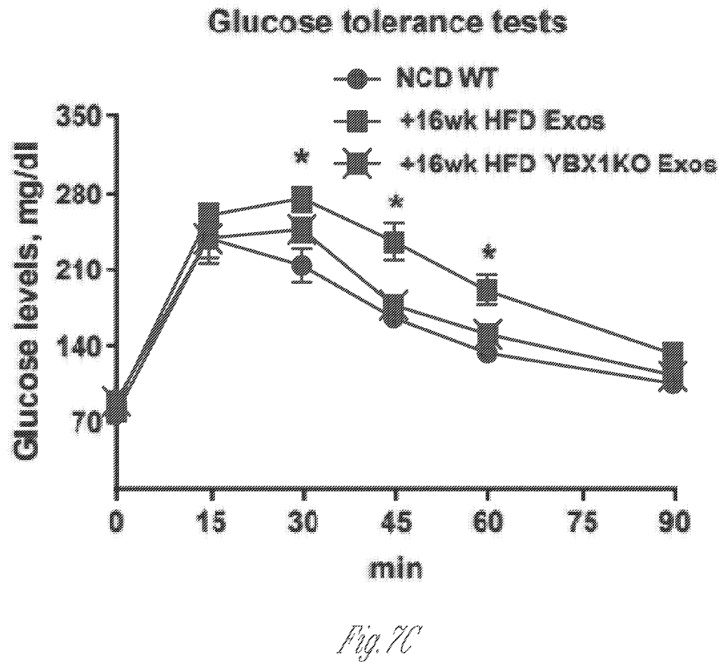

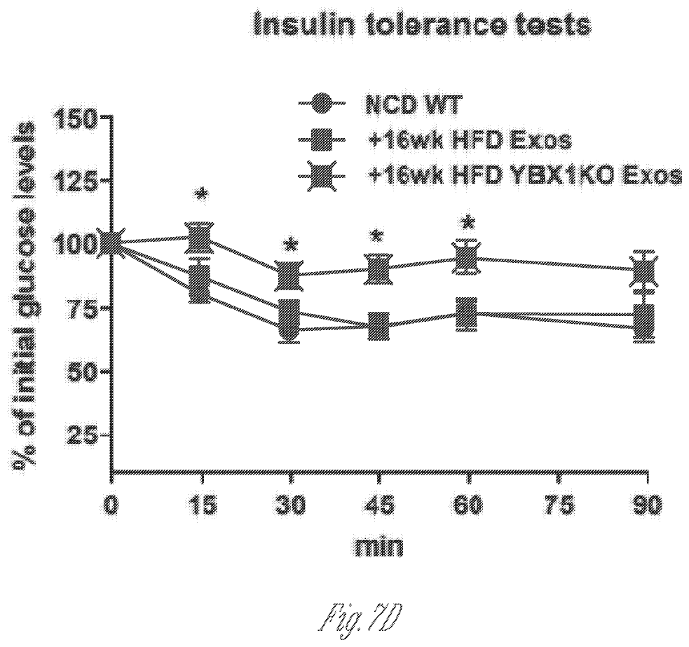

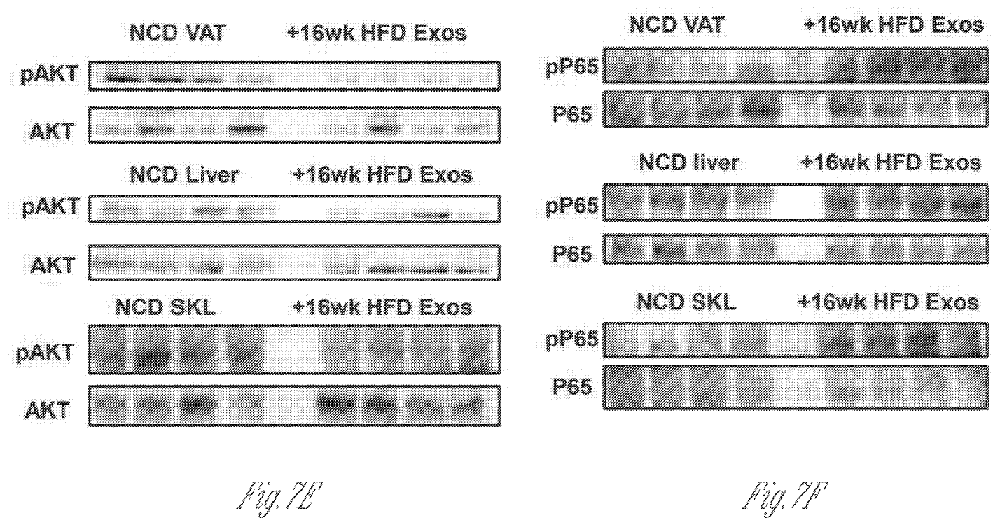

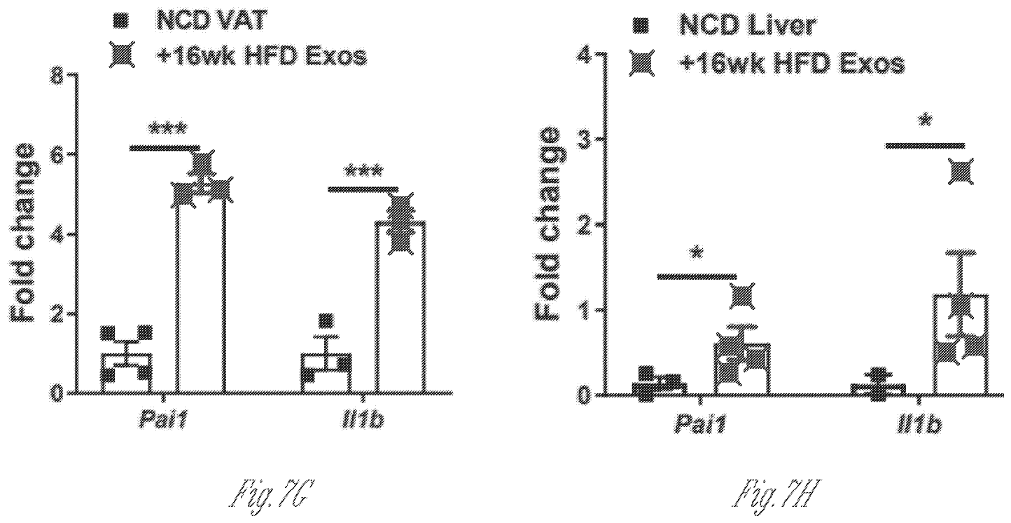

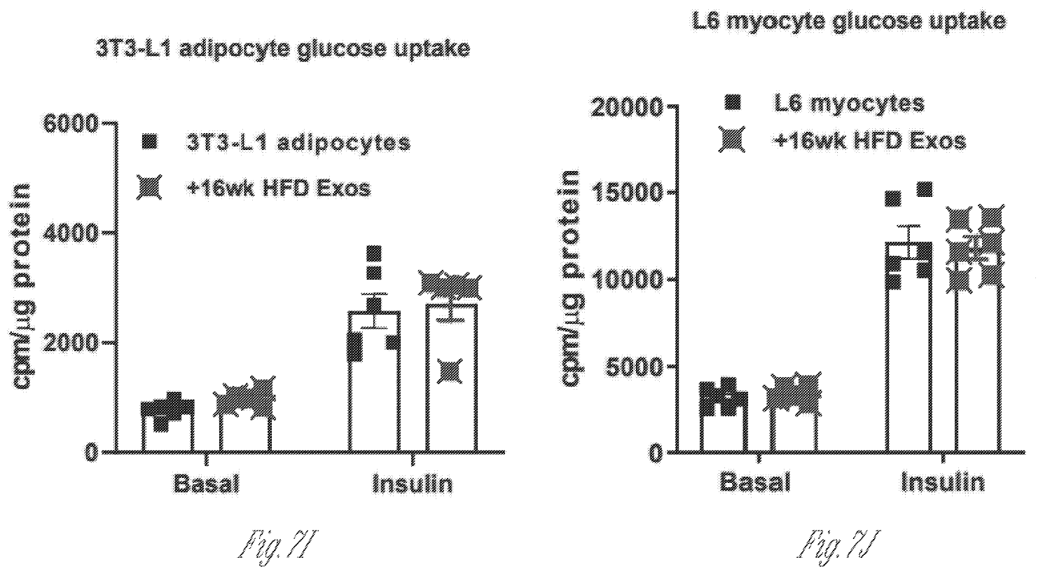

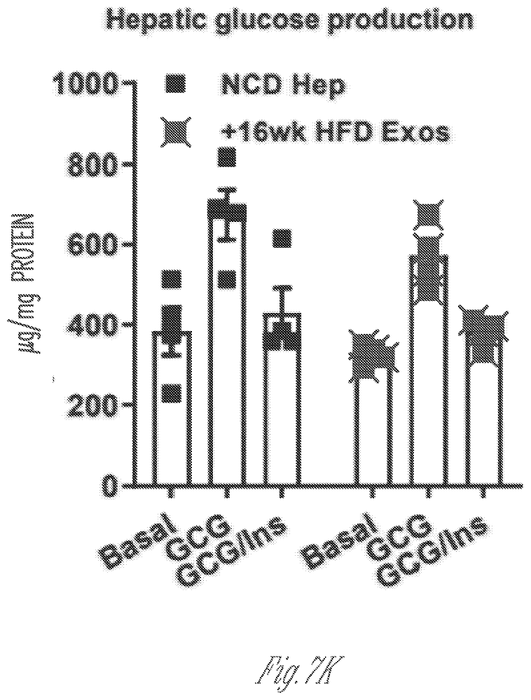

Depletion of either Rab27 or YBX1 in hepatocytes led to improved glucose and insulin tolerance compared to 16 week HFD WT mice injected with a lentiviral control vector (FIG. 7A-7B). Indeed, this beneficial effect was quite profound, because only intermediate levels of glucose and insulin tolerance were observed in these hepatocyte exosomal miRNA depleted mice—levels that were between chow-fed and 4 week HFD mice.

To test the effect of Exos derived from these mouse models on insulin sensitivity, lean wild type mice were intravenously injected with Exos derived from either 16 week HFD wild type or 16 week HFD YBX1-KO hepatocytes (1×10⁹ Exos/mouse). After 5 weeks treatment with 16 week HFD/obese wild type Exos, lean recipient mice displayed decreased glucose tolerance and insulin sensitivity, whereas, miRNA-free 16 week HFD/obese YBX1KO Exos had negligible effects (FIG. 7C-7D). In addition, 16 week HFD/obese hepatocyte Exos treatment caused decreased levels of insulin-stimulated AKT phosphorylation in key metabolic tissues of lean recipient mice, compared to controls (FIG. 7E). Interestingly, 16 week HFD/obese Exos did not directly affect cellular insulin sensitivity, as evidenced by the lack of an in vitro effect of these Exos on cellular insulin responses in 3T3-L1 adipocytes, L6 myocytes, and lean primary hepatocytes (FIG. 7I-7K).

Tissue inflammation is a hallmark of obesity and inflammation exacerbates insulin resistance. Treatment of lean mice 16 wk HFD/obese Exos led to tissue inflammation, as shown by increased activation of the NFκB pathway and proinflammatory cytokine expression in adipose tissue, liver, and skeletal muscle (FIG. 7F-7H).

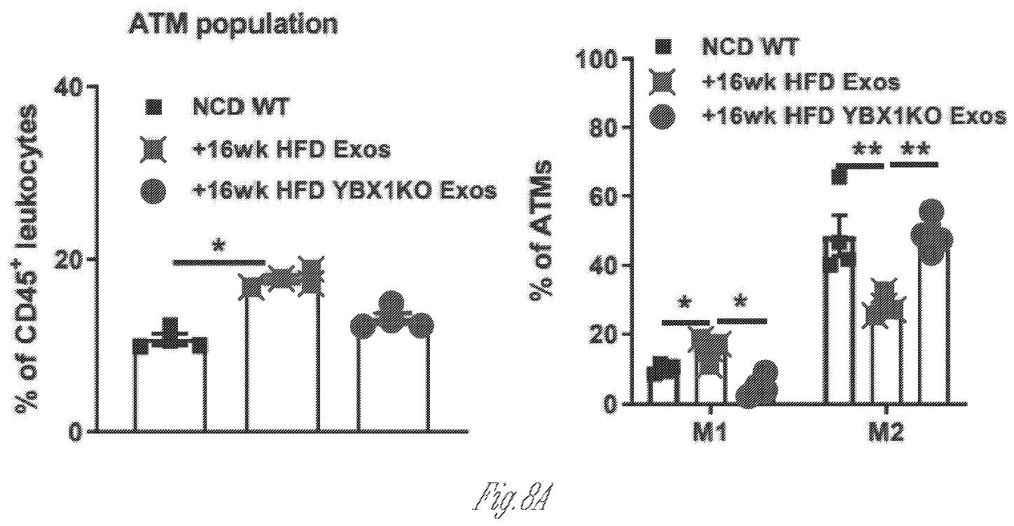

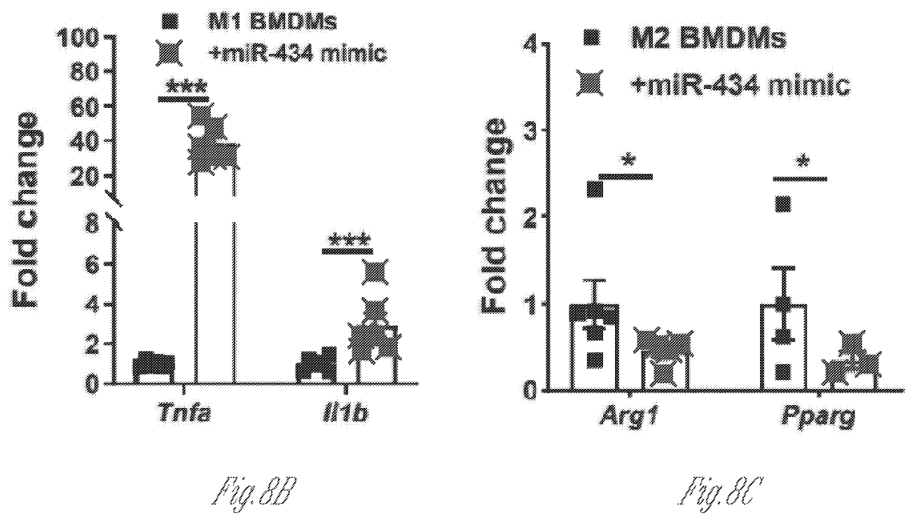

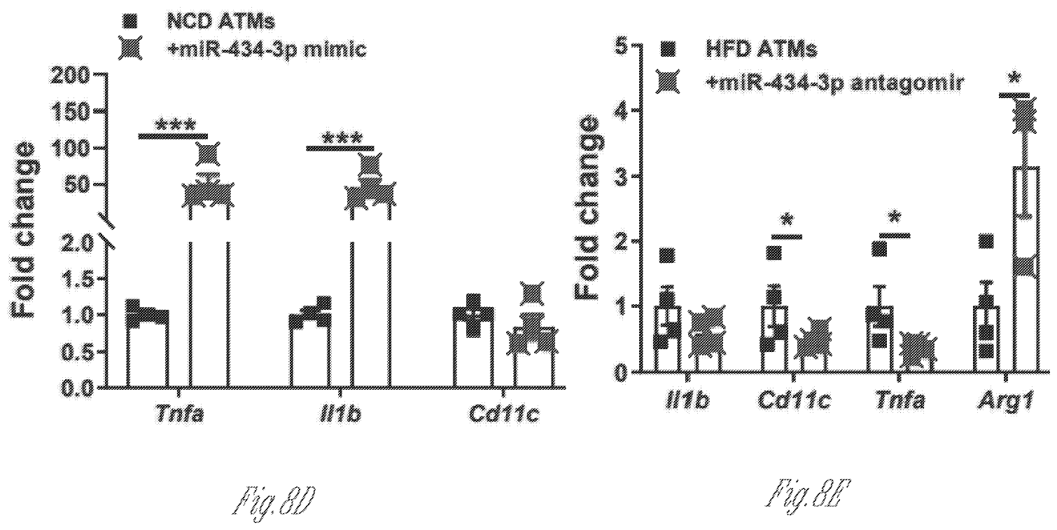

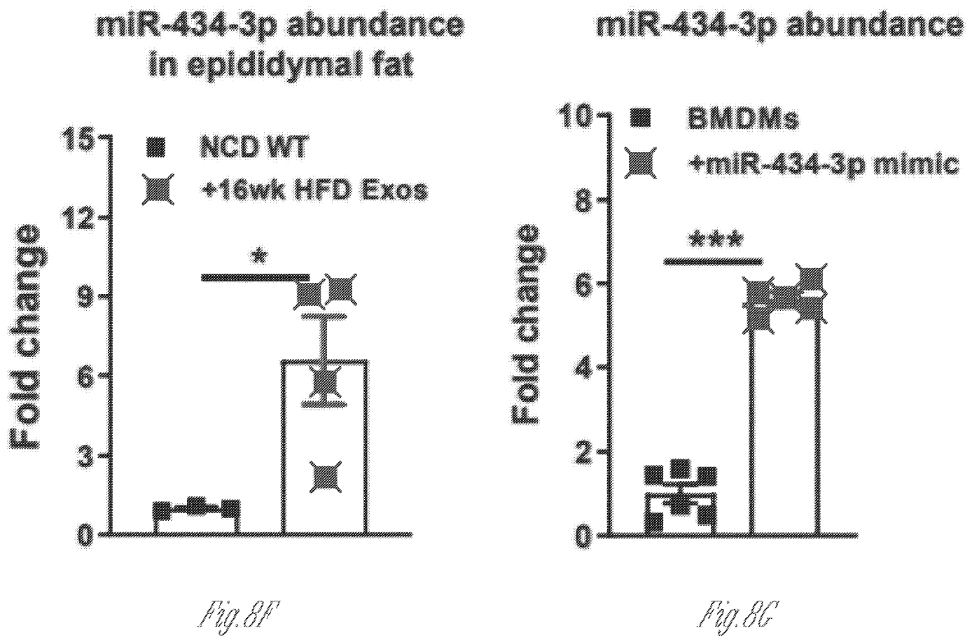

Given that 16 week HFD/obese Exos treatment enhanced tissue inflammation, the population and activation of adipose tissue macrophages (ATMs) was also examined. Flow cytometric analysis showed that treatment with 16 week HFD/obese Exos led to greater proinflammatory (CD11c+CD206-F4/80+CD11b+) ATMs accumulating in eWAT, compared to control lean mice treated with empty liposomes (FIG. 8A). In contrast, in vivo treatment with miRNA-free 16 week HFD/obese YBX1-KO Exos treatment did not affect the ATM population or activation state, demonstrating the necessity of the Exos miRNA cargo for the effects of 16 week HFD/obese Exos (FIG. 8A). Thus, these results indicate that 16 wk HFD/obese Exos can indirectly contribute to insulin resistance by promoting obesity-associated tissue inflammation.

### Example 7: miR-434-3p Promotes Macrophage Proinflammatory Activation

The effect of miRNAs enriched 16 week HFD/obese hepatocyte Exos on macrophage activation was assessed. Exos miRNA sequencing showed that miR-434-3p was one of the highly expressed miRNAs in 16 week HFD Exos.

Experiments showed that miR-434-3p abundance was increased in the eWAT of NCD mice after 5 weeks treatment with 16 week HFD/obese hep Exos (FIG. 8F). More importantly, treatment with liposomes containing a miR-434-3p mimic enhanced the proinflammatory phenotype of LPS-stimulated M1 bone marrow-derived macrophages (BMDMs) and repressed the anti-inflammatory activation of IL4/IL13-induced M2 BMDMs (FIG. 8B-8C, 8G). In addition, transfection of NCD ATMs with the miR-434-3p mimic led to increased expression of proinflammatory cytokines compared to cells transfected with a negative miRNA mimic control (FIG. 8D). Treatment with a miR-434-3p antagomir attenuated the proinflammatory activation of ATMs isolated from obese mice (FIG. 8E). Consistent with the lack of direct effects of 16 week HFD/obese Exos on cellular insulin signaling, transfection of liposome-containing the miR-434-3p mimic did not affect in vitro cellular insulin responses.

In summary, it was shown that during the course of obesity hepatocyte-derived Exos have important effects to modulate systemic glucose tolerance and insulin sensitivity. Early in the onset of obesity, hepatocytes secrete Exos that promote an insulin-sensitive state, presumably as a compensatory response to the caloric excess which leads to the opposite effect by causing insulin resistance. These beneficial properties of 4 week HFD Exos are mediated through their miRNA cargo. MiR-3075 was identified as the dominant insulin-sensitizing molecule within these Exos. Treatment with a miR-3075 mimic led directly to improved insulin signaling in vitro, and when given in vivo, caused improved glucose tolerance and heightened insulin sensitivity. As the obesity process progresses to the chronic state, these insulin-sensitizing Exos are no longer produced, and, instead, hepatocytes now make Exos containing miRNAs (e.g., miR-434-3p) that directly promote insulin resistance and glucose intolerance by stimulating proinflammatory polarization of ATMs. Therefore, the miR-3075 can be used as an insulin-sensitizing therapeutic. In addition, an antagomir directed against miR-434-3p can also have beneficial effects.

### REFERENCES

Agarwal et al., Elife, 4:_(2015).
Auberger et al., Cell, 58:631 (1989).
Baek et al., Nature, 455:64 (2008).
Bartel, Cell, 136:215 (2009).
Bobrie et al., Cancer Res., 72:4920 (2012).
Castano et al., Proc. Natl. Acad. Sci. USA, 115:12158 (2018).
Chavez et al., Diabetes Care, 32:1542 (2009).
Chen et al., Am. J. Physiol. Gastrointest. Liver Physiol., 309:G491 (2015).
Chen et al., Hepatol. Commun., 3:180 (2019).
Choi et al., Diabetes Metab. J., 37:63 (2013).
Deng et al., Diabetes, 58:2498 (2009).
Ebert et al., Metabolism., 58:547 (2009).
Ferrante et al., Pediatr. Res., 77:447 (2015).
Flaherty et al., Science, 3_6:989 (2019).
Gao et al., Mol. Metab., 4:310 (2015).
Ghorpade et al., Nature, 555:673 (2018).
Kahn et al., Nature, 444:840 (2006).
Kharitonenkov et al., J. Clin. Invest., 115:1627 (2005).
Kumar et al., Cell Metab., 8:468 (2008).
Lee et al., Cell, 172:772 (2018).
Lee et al., Sci. Rep-Uk, 7: (2017).
Li et al., Cell, 167:973 (2016).
Mathieu et al., Nat. Cell Biol., 21:9 (2019).
Misu et al., Cell. Metab., 12:483 (2010).
Mori et al., Cell. Metab., __:__(2019).
Mukhopadhyay & Bhattacharya, Diabetologia, 59:859 (2016).
Nesto et al., Diabetes Care, 27:256 (2004).
Oike et al., Nat. Med., 11:400 (2005).
Ostrowski et al., Nat. Cell. Biol., 12:19 (2010).
Pescador et al., PLoS One, 8:e77251 (2013).
Platt et al., Cell, 159:440 (2014).
Povero et al., Cell. Mol. Gastroenterol. Hepatol., 1:646 (2015).
Ran et al., Nature, 520:186 (2015).
Roden & Shulman, Nature, 576:51 (2019).
Romeo et al., Arterioscler. Thromb. Vasc. Biol., 32:1771 (2012).
Scheja & Heeren, J. Hepatol., 64:1176 (2016).
Selbach et al., Nature 455:58 (2008).
Shurtleff et al., Elife, 5:_(2016).
Shurtleff et al., Proc. Natl. Acad. Sci. USA, 1_14:E8987 (2017).
Song et al., Nat. Commun., 10:1639 (2019).
Thomou et al., Nature, 542:450 (2017).
Turner et al., Diabetologia, 56:1638 (2013).
Varin et al., Cell. Metab., 29:320 (2019).
Wu et al., Diabetologia, 59:1732 (2016).
Yang et al., J. Clin. Endocrinol. Metab., 96:E1325 (2011).
Ying et al., Cell, 171:372 (2017).
Ying et al., Cell. Metab., ____(2021).
Ying et al., J. Vis. Exp., __:_(2013).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

The following statements summarize some of the aspects of the invention.

### Statements

1. A method to enhance insulin sensitivity, reduces obesity, or a combination thereof in a mammal, comprising: administering to a mammal in need thereof an effective amount of a composition comprising one or more nucleic acids, each having a sequence comprising at least a seed region of miRNA-3075-5p.
2. The method of statement 1, wherein the composition further comprises one or more agents that inhibit miR-434-3p.
3. The method of statement 2, wherein one or more agents that inhibit miR-434-3p comprises a nucleic acid having sequence complementary to 6-9 contiguous nucleotides of an endogenous miR-434-3p RNA seed sequence.
4. The method of statement 3, wherein the endogenous miR-434-3p RNA sequence comprises at least 90% sequence identity to SEQ ID NO:4 or SEQ ID NO:5.
5. The method of any one of statements 1 to 4, wherein mammal has Type 2 Diabetes Mellitus, Nonalcoholic liver disease (NASH), polycystic ovarian syndrome, or obesity, or is at risk for development of Type 2 Diabetes Mellitus, Nonalcoholic liver disease (NASH), polycystic ovarian syndrome, or obesity.
6. The method of any one of statements 1 to 5, which decreases insulin resistance in a mammal.
7. The method of any one of statements 1 to 6, which decreases obesity in the mammal.
8. The method of any one of statements 1 to 7, wherein the mammal is a human.
9. The method of any one of statements 1 to 8, wherein the composition comprises liposomes.
10. The method of statement 9, wherein the liposomes comprise or more of DC-cholesterol, 1,2-dioleoyl-sn-glycero-3-phosphoethanolamine (DOPE) an ionizable cationic lipid or a lipidoid.
11. The method of any one of statements 1 to 10, wherein the composition comprises nanoparticles.
12. The method of any one of statements 1 to 11, wherein the composition is systemically administered.
13. The method of any one of statements 1 to 12, wherein the composition is orally administered.
14. The method of any one of statements 1 to 12, wherein the composition is injected.
15. The method of any one of statements 1 to 14, wherein the seed region of miRNA-3075-5p comprises 5′CUGGGAG3′ or 5′GCCAAGGA3′.
16. The method of any one of statements 1 to 15, wherein the miRNA-3075-5p comprises at least 95% sequence identity or complementarity to SEQ ID NO:3.
17. The method of any one of statements 1 to 16, wherein the miRNA-3075-5p is a miRNA-3075 microRNA comprising at least 90% sequence identity or complementarity to SEQ ID NO:1 or SEQ ID NO:2.
18. The method of any one of statements 1 to 17, wherein the nucleic acid is less than 30 bases in length.
19. The method of any one of statements 1 to 18, wherein the nucleic acid is less than 25 bases in length.
20. The method of any one of statements 1 to 19, wherein the nucleic acid is less than 20 bases in length.
21. The method of any one of statements 1 to 20, wherein the nucleic acid is greater than 10 bases in length.
22. The method of any one of statements 1 to 21, wherein the composition comprises single stranded RNAs, each comprising a miRNA-3075-5p or miR-434-3p seed region, or a complement thereof.
23. The method of any one of statements 1 to 22, wherein the composition comprises RNA comprising a hairpin-loop structure.
24. The method of any one of statements 1 to 23, wherein the composition comprises one or double stranded nucleic acids, each comprising the seed region.
25. The method of any one of statements 1 to 24, wherein the RNA or one strand of the double stranded nucleic acid is less than 70 bases in length.
26. The method of any one of statements 1 to 25, wherein the RNA or one strand of the double stranded nucleic acid is less than 50 bases in length.
27. The method of any one of statements 1 to 26, wherein the RNA or one strand of the double stranded nucleic acid is less than 40 bases in length.
28. The method of any one of statements 1 to 27, wherein the RNA or one strand of the double stranded nucleic acid is less than 25 bases in length.
29. The method of any one of statements 1 to 28, wherein the RNA or one strand of the double stranded nucleic acid is greater than 10 bases in length.
30. The method of any one of statements 1 to 29, wherein the length of the one strand is greater than the length of the seed region.
31. The method of any one of statements 1 to 30, wherein the composition comprises one or more nucleic acid sequences comprising non-native nucleotides.
32. The method of any one of statements 1 to 31, wherein the composition comprises one or more nucleic acids comprising at least one modified nucleobase, modified phosphate group, modified sugar, or a combination thereof.
33. The method of any one of statements 1 to 32, wherein the amount of the nucleic acid is about 0.01 mg/kg to about 100 mg/kg.
34. The method of any one of statements 1 to 33, wherein the amount of the nucleic acid is about 0.05 mg/kg to about 10 mg/kg.
35. The method of any one of statements 1 to 34, wherein the amount of the nucleic acid is about 10 mg/kg to about 75 mg/kg.
36. The method of any one of statements 1 to 35, wherein the amount of the nucleic acid is about 1 mg/kg to about 100 mg/kg.
37. A composition comprising one or more nucleic acids, each having a sequence comprising at least a seed region of miRNA-3075-5p.
38. The composition of statement 37, wherein the composition further comprises one or more agents that inhibit miR-434-3p.
39. The composition of statement 37 or 38, which comprises one or more nucleic acids encapsulated in liposomes.
40. The composition of statement 39, wherein the liposomes comprise or more of DC-cholesterol, 1,2-dioleoyl-sn-glycero-3-phosphoethanolamine (DOPE) an ionizable cationic lipid or a lipidoid.
41. The composition of statement 37 or 38, wherein the composition comprises nanoparticles.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method to enhance insulin sensitivity, reduce obesity, or a combination thereof in a mammal, comprising: administering to a mammal in need thereof an effective amount of a composition comprising one or more nucleic acids, each having a sequence comprising at least a seed region of miRNA-3075-5p.

2. The method of claim 1, wherein the composition further comprises one or more agents that inhibit miR-434-3p.

3. The method of claim 2, wherein one or more agents that inhibit miR-434-3p comprises a nucleic acid having sequence complementarity to 6-9 contiguous nucleotides of an endogenous miR-434-3p RNA seed sequence.

4. The method of claim 3, wherein the endogenous miR-434-3p RNA sequence comprises at least 90% sequence identity to SEQ ID NO:4 or SEQ ID NO:5.

5. The method of claim 1, wherein mammal has Type 2 Diabetes Mellitus, Nonalcoholic liver disease (NASH), polycystic ovarian syndrome, or obesity, or is at risk for development of Type 2 Diabetes Mellitus, Nonalcoholic liver disease (NASH), polycystic ovarian syndrome, or obesity.

6-8. (canceled)

9. The method of claim 1, wherein the composition comprises liposomes.

10. (canceled)

11. The method of claim 1, wherein the composition comprises nanoparticles.

12-14. (canceled)

15. The method of claim 1, wherein the seed region of miRNA-3075-5p comprises 5′CUGGAG3′ or 5′GCCAAGGA3.

16. The method of claim 1, wherein the miRNA-3075-5p comprises at least 95% sequence identity or complementarity to SEQ ID NO:3.

17. The method of claim 1, wherein the miRNA-3075-5p is a miRNA-3075 microRNA comprising at least 90% sequence identity or complementarity to SEQ ID NO:1 or SEQ ID NO:2.

18-21. (canceled)

22. The method of claim 1, wherein the composition comprises single stranded RNAs, each comprising a miRNA-3075-5p or miR-434-3p seed region, or a complement thereof.

23. The method of claim 1, wherein the composition comprises RNA comprising a hairpin-loop structure.

24. The method of claim 1, wherein the composition comprises one or double stranded nucleic acids, each comprising the seed region.

25-29. (canceled)

30. The method of claim 1, wherein one or more nucleic acids comprises a double stranded nucleic acid where the length of the one strand is greater than that of the nucleic acid sequence having the seed region.

31-32. (canceled)

33. The method of claim 1, wherein the amount of the nucleic acid is about 0.01 mg/kg to about 100 mg/kg.

34-36. (canceled)

37. A composition comprising one or more nucleic acids, each having a sequence comprising at least a seed region of miRNA-3075-5p.

38. The composition of claim 37, wherein the composition further comprises one or more agents that inhibit miR-434-3p.

39. The composition of claim 37, which comprises one or more nucleic acids encapsulated in liposomes.

40. The composition of claim 38, comprising one or more of the agents that inhibit miR-434-3p encapsulated in liposomes.

41. (canceled)

42. The composition of claim 37, wherein the composition comprises nanoparticles.

43. The composition of claim 38, comprising one or more of the agents that inhibit miR-434-3p as nanoparticles.


SEQUENCE LISTING (15 sequences)

SEQ ID NO: 1 - RNA, 21 nt, Homo sapiens
ugucugggagcagccaaggac

SEQ ID NO: 2 - RNA, 85 nt, Homo sapiens
ugugguuuguacugucugggagcagccaaggacaaguuaccucuugucuucuauccuuggccuuccuagguguccaagcucacag

SEQ ID NO: 3 - RNA, 39 nt, Homo sapiens
ugugguuuguacugucugggagcagccaaggacaaguua

SEQ ID NO: 4 - RNA, 22 nt, Homo sapiens
uuugaaccaucacucgacuccu

SEQ ID NO: 5 - RNA, 94 nt, Homo sapiens
ucgacucuggguuugaaccaaagcucgacucaugguuugaaccauuacuuaauucgugguuugaaccaucacucgacuccugguucgaaccauc

SEQ ID NO: 6-14 - skipped (9 entries)

SEQ ID NO: 15 - RNA, 9 nt, Artificial Sequence
  A synthetic sequence
uucaagaga